(19) United States
(12) Patent Application Publication
(10) Pub. No.: US 2024/0238426 A1
(43) Pub. Date: Jul. 18, 2024

(54) PROTEIN ACYLATION MODULATION BY HETEROBIFUNCTIONAL MOLECULES

(71) Applicant: THE SCRIPPS RESEARCH INSTITUTE, La Jolla, CA (US)

(72) Inventors: Christopher Parker, Palm Beach Gardens, FL (US); Wei Wang, Jupiter, FL (US); Li Yun Chen, Palm Beach Gardens, FL (US)

(21) Appl. No.: 18/548,630

(22) PCT Filed: Mar. 4, 2022
(86) PCT No.: PCT/US22/18925

§ 371 Date: Sep. 1, 2023

Related U.S. Application Data
(60) Provisional application No. 63/156,571, filed on Mar. 4, 2021.

(51) Int. Cl. A61K 47/55; A61K 47/54

(52) U.S. Cl. CPC A61K 47/55; A61K 47/542; A61K 47/545

(57) ABSTRACT

Compositions and methods for control and/or modification of endogenous protein acetylation are described. The compositions are directed to heterobifunctional molecules having protein and enzyme binding moieties linked together by an organic linker group. The compositions are selective for binding to certain endogenous proteins and function to recruit acety lation enzymes to acetylate or deacety late the proteins.

### CROSS-REFERENCE TO RELATED APPLICATIONS

The subject patent application claims the benefit of priority to U.S. Provisional Patent Application No. 63/156,571 (filed Mar. 4, 2021; now pending). The full disclosure of the priority application is incorporated herein by reference in its entirety and for all purposes.

### SEQUENCE LISTING

This application incorporates by reference a Sequence Listing in the form of a ST.25 text file labeled “Sequence Listing_ST25”. The file is of 3 KB and was created on Nov. 4, 2022.

BACKGROUND

Protein acetylation is the second most frequent post-translation modifications (PTMs) found in the human proteome, occurring on >20,000 sites, Khoury, et al. ^{[1]}. Protein acetylation has profound effects on protein function, which regulates diverse cellular processes ^{[2]}. Lysine acetyltransferase (KATs) and lysine deacetylase (KDACs) maintain dynamic acetylation levels on lysine residues ^{[3]}. KDAC inhibitors, such as Vorinostat (SAHA) have been clinically approved to treat diseases, such as T-cell lymphoma ^{[4]}. However, given that thousands of acetylation sites are modified by a combined ˜40 KATs and KDACs, blockade of their enzymatic activity represents a non-specific method to investigate their roles and treat diseases^{[5]}.

Therefore, a goal of the present invention is the development of a method for selective control, and/or modification and/or blockage of KATs and KDACs activity. A further goal is the use of a selective control/modification method for treatment of abnormal cellular activity such as exhibited by non-differentiated aplastic cells, and in particular neoplastic or cancer cells.

### SUMMARY

Aspects of the present invention are directed to embodiments of a method for the selective control of protein acetylation and to embodiments of compositions for practice of the method. A further aspect of this the invention is directed to development of a series of heterobifunctional molecules designed to modulate targeted protein acetylation.

Embodiments of the composition aspect of the invention are directed to heterobifunctional molecules having a protein binding terminus (moiety) (PBT) linked by a linking moiety (L) to a recruiter binding terminus (moiety) RBT for an acetylase enzyme. The protein binding moiety may be an organic small molecule moiety having a selective binding engagement with a selected endogenous protein. The recruiter binding moiety may be an organic small moiety that has the capacity to recruit and bind an acetylation enzyme in a milieu containing the endogenous acetylation enzyme. The heterobifunctional molecules designed to bind selectively certain proteins and recruit acetylation enzymes are referred to herein as AceTAG and DeAceTAG which are acronyms for acetylation tagging and deacetylation tagging.

A representative embodiment of an AceTAG/DeAceTag may be configured as Formula I:

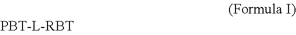

wherein PBT is a small organic moiety capable of selectively binding to a preferably endogenous protein present in a mixture of differing preferably endogenous proteins, L is an organic linker and RBT is a small organic moiety capable of recruiting and binding to a preferably endogenous acetylase enzyme present in a mixture of preferably endogenous enzymes.

Embodiments of the (De)AceTAG heterobifunctional molecule of Formula I and the method for their enzymatic and cellular activity are depicted in FIG. 1 (synonymous description of Formula I being TBT-L-EBT of FIG. 1 as explained below). Focused embodiments of the AceTAG of Formula I may be designed to bind with the FKBP12^{F36V} protein and to recruit the p300/CBP enzyme in vivo. A subgeneric formula of Formula I designed in this fashion may be Formula II which depicts the generic formulation of the linker L and specific structures of the PBT and RBT. Formula II is also depicted in FIG. 2A.

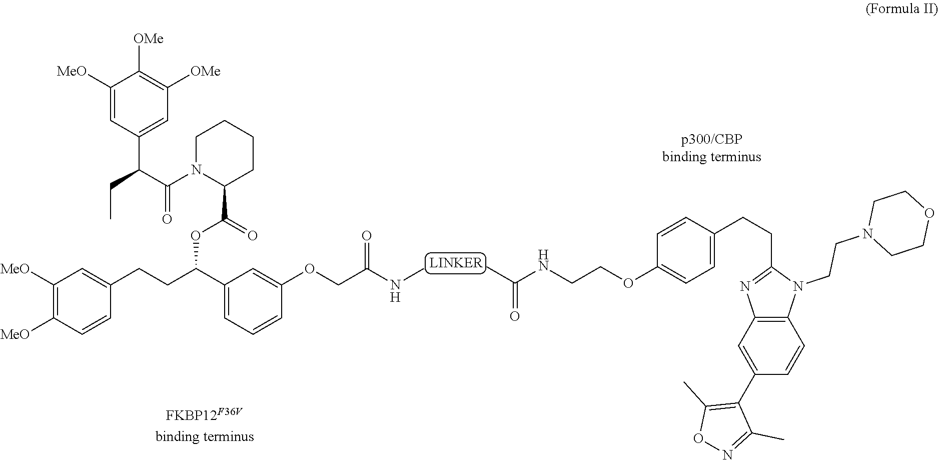 [SMILES: CC[C@H](C(=O)N1CCCC[C@H]1C(=O)O[C@@H](CCc1ccc(OC)c(OC)c1)c1cccc(OCC(=O)NC)c1)c1cc(OC)c(OC)c(OC)c1 ; CC(=O)NCCOc1ccc(CCc2nc3cc(-c4c(C)noc4C)ccc3n2CCN2CCOCC2)cc1]

More specific structures of Formula II as AceTAG-1 through AceTAG-4 are depicted in FIG. 2B. These depicted structures have the same PBT and RBT moieties which respectively are a di-aromatically substituted piperidinylcarboxylpropyl phenyl glycolate ester moiety and a 2-(hydroxyphenylethyl)-1-(morpholinylethyl)5-oxazolyl-benzimidazole moiety. The linker L is the variable and may be a polyol, a polyamide or an alkylenyl moiety of 2 or more units up to an oligomeric length. The linker may have amine and/or carboxyl termini for binding the PBT and RBT moieties to the linker.

Further aspects of the present invention are directed to methods for modulation of acetylation of endogenous proteins in vitro and in vivo. Embodiments of these aspects involve use of the AceTAG and DeAceTAG heterobifunctional molecular embodiments (hereinafter together (De)AceTAG) in vitro enzyme media, in vivo cellular cultures and whole organisms such as mammals to investigate acetylation on protein function and to pursue therapeutic aspects for treatment of disease. Embodiments of therapeutic aspects include but are not limited to treatment of neoplastic and/or diseased and/or cancer cells through interdiction of the p53 cellular pathway.

### BRIEF DESCRIPTION OF DRAWINGS

FIG. 1 provides a schematic showing how heterobifunctional small molecules of Formula I can precisely regulate acetylation on proteins of interest (POIs). This method employing AceTAG/DeAceTAG of Formula I represents a powerful strategy to investigate PTM function and may yield strategies to treat human disease. These bifunctional molecules include protein target-binding terminus or moiety that is capable of binding selectively to a POI (PBT which is signified as TBT, target binding terminus on FIG. 1), a linker (L) and a recruiter binding terminus or moiety that is capable of recruiting and binding to an acetylation enzyme (KAT or KDAC, RBT which is signified as EBT, editor binding terminus in FIG. 1). The heterobifunctional molecules enable proximity induced modification of the POI by acetylation/deacetylation.

FIGS. 2A and 2B illustrate the design of AceTAG molecules to chemically induce proximity of p300/CBP and protein of interest. FIG. 2A provides a schematic depiction of the AceTAG system. Heterobifunctional molecules induce dimerization of FKBP12^{F36V} fusion protein and the transcriptional co-activator p300, leading to p300-mediated acetylation. FIG. 2B provides chemical structures of p300/CBP ligand coupled to a FKBP12^{F36V} ligand.

FIGS. 3A, 3B, 3C, 3D and 3E provide a schematic depiction of AlphaScreen assay and AlphaScreen analyses as well as immunoblot analyses of ternary complex formation. FIG. 3A illustrates a schematic depiction of AlphaScreen assay. FIG. 3B depicts the AlphaScreen analysis of AceTAG-mediated ternary complex formation between FKBP12^{F36V} and the bromodomain of p300 (BRD-p300). FIG. 3C depicts the AlphaScreen analysis of competition between AceTAG and increasing concentrations of p300-binding ligand. FIG. 3D depicts the AlphaScreen analysis of competition between AceTAG and increasing concentrations of FKBP12^{F36V}-binding ligand. FIG. 3E depicts the immunoblot analysis of FKBP12^{F36V} photo-crosslinking probe binding in HEK293T cells. Structure of FKBP12^{F36V} photo-crosslinking probe. FIG. 3F depicts the immunoblot analysis of p300/CBP photo-crosslinking probe binding in HEK293T cells. Structure of p300/CBP photo-crosslinking probe.

FIGS. 4A, 4B, 4C, 4D, 4E, and 4F provide a schematic and immunoblot analyses of H3.3-FKBP12^{F36V} HeLa cell line treated with AceTAG heterobifunctional molecules as depicted in FIG. 2. The AceTAG molecules induce p300-mediated acetylation of the protein H3.3-FKBP12^{F36V}. FIG. 4A provides a schematic depiction of lentivirus expression strategy in HeLa, H1299, and HeLa RelA−/− cell lines. FIG. 4B depicts the immunoblot analysis of H3.3-FKBP12^{F36V} HeLa cells treated with DMSO and AceTAG-1 at the indicated doses for 2 hr. Acetylation of K18, K23, and K27 were monitored. FIG. 4C depicts the immunoblot analysis of H3.3-FKBP12^{F36V} HeLa cells treated with DMSO and AceTAG-1 at the indicated doses for 2 hr. Acetylation of K9, K14, and K79 were monitored. FIG. 4D depicts the immunoblot analysis of H3.3-FKBP12^{F36V} HeLa cells treated with DMSO, 625 nM AceTAG-1, 5 μM SAHA, 625 nM AceTAG-1 with 50 μM FKBP ligand, and 625 nM AceTAG-1 with 1 μM A485 for 2 hr. FIG. 4E depicts the immunoblot analysis of H3.3-FKBP12^{F36V} HeLa cells treated with DMSO and 625 nM AceTAG-1 for the indicated time. FIG. 4F depicts the immunoblot analysis of washout experiments in H3.3-FKBP12^{F36V} HeLa cells.

FIGS. 5A, 5B, 5C, 5D, and 5E provide quantitative proteomics analysis of selectivity of AceTAG-induced H3.3-FKBP12^{F36V} acetylation. FIG. 5A depicts quantitative proteomics analysis of AceTAG-induced H3.3-FKBP12^{F36V} acetylated lysine abundances. H3.3-FKBP12^{F36V} HeLa cells treated with DMSO, 625 nM AceTAG-1, or 5 μM SAHA. Data represent a median of n=6 biological replicates for AceTAG-1-treated cells and a median of n=2 biological replicates for SAHA-treated cells. FIG. 5B depicts immunoblot analysis of H3.3-FKBP12^{F36V} HeLa cells treated with increasing concentration of AceTAG. Acetylated c-Myc and acetylated STAT3 were monitored. FIG. 5C depicts immunoblot analysis of H3.3-FKBP12^{F36V} Hela cells treated with increasing concentration of AceTAG. Broad acetylated lysine changes were monitored. FIG. 5D depicts acetylproteomics analysis of H3.3-FKBP12^{F36V} Hela cells treated with DMSO and 600 nM AceTAG. FIG. 5E depicts acetylproteomics analysis of H3.3-FKBP12^{F36V} HeLa cells treated with DMSO and 2 μM SAHA. The vertical dashed lines correspond to 2-fold change in enrichment relative to DMSO, and the horizontal line corresponds to a P value of 0.05 for statistical significance. Red circles correspond to protein targets with >2-fold change (P<0.05) relative to DMSO. Each point represents an individual acetylated peptide plotted as a mean of n=3 biological replicates for DMSO and AceTAG-1 treatments and n=2 biological replicates for SAHA treatments combined in one TMT 10-plex experiment.

FIGS. 6A, 6B, 6C, and 6D provide immunoblot analyses of heterobifunctional molecules of FIGS. 2A and 2B applied to induce p300-mediated acetylation of RelA and p53. FIG. 6A shows an immunoblot analysis of FKBP12^{F36V}-RelA RelA−/− HeLa cells treated with DMSO and AceTAG-1 at the indicated doses for 2 hr. Acetylation of K310 was monitored. FIG. 6B shows an immunoblot analysis of FKBP12^{F36V}-RelA RelA−/− HeLa cells treated with DMSO, 625 nM AceTAG-1, 5 μM SAHA, 625 nM AceTAG-1 with 50 μM FKBP ligand, and 625 nM AceTAG-1 with 1 μM A485 for 2 hr. FIG. 6C shows an immunoblot analysis of FKBP12^{F36V}-p53 H1299 cells treated with DMSO and AceTAG-1 at the indicated doses for 2 hr. Acetylation of K305, K373, and K382 were monitored. FIG. 6D shows an immunoblot analysis of FKBP12^{F36V}-p53 H1299 cells treated with DMSO, 625 nM AceTAG-1, 5 μM SAHA, 625 nM AceTAG-1 with 50 μM FKBP ligand, and 625 nM AceTAG-1 with 1 μM A485 for 2 hr.

FIGS. 7A, 7B, 7C, 7D, 7E, 7F, and 7G provide results from immunoblot analysis, qPCR, RNA-seq, and ChIP-qPCR studies of AceTAG-medidated p53 target genes activation. FIG. 7A shows immunoblot analyses of p21, PUMA, GLS2, and MDM2 expression level in FKBP12^{F36V}-p53 H1299 cells treated with DMSO and AceTAG-1 at the indicated doses. FIG. 7B shows an immunoblot analysis of p21 expression level in FKBP12^{F36V}-p53 H1299 cells treated with DMSO and AceTAG-1 at the indicated doses for 24 hr. FIG. 7C shows an qPCR analysis of p21 mRNA level in FKBP12^{F36V}-p53 H1299 cells treated with DMSO and AceTAG-1 at the indicated doses for 8 hr. FIG. 7D shows an immunoblot analysis of p21 expression level in both p53 H1299 and FKBP12^{F36V}-p53 H1299 cells treated with DMSO and AceTAG molecules at the indicated doses for 8 hr. FIG. 7E shows and immunoblot analysis of p21 expression level in FKBP12^{F36V}-p53 H1299 cells treated with DMSO, 3 μM AceTAG-1, 3 μM p300/CBP-binding ligand (p300-c), 3 μM FKBP12^{F36V}-binding ligand (FKBP-c), 3 μM AceTAG-1 with 10 μM A485, and 3 μM AceTAG-1 with 50 μM FKBP12^{F36V}-binding ligand for 8 hr. FIG. 7F shows RNA-seq analysis of both H1299 and FKBP12^{F36V}-p53 H1299 cells treated with DMSO and 3 μM AceTAG-1. FIG. 7G shows ChIP-qPCR analysis of FKBP12^{F36V}-p53 H1299 cells treated with DMSO and 3 pM AceTAG-1. Data represents a median of n=3 biological replicates.

FIG. 8 provides a blot graph of the results of acetylation of FKBP12^{F36v} transfected into HeLa cells and treated with the AceTAG examples AceTAG-1, AceTAG-2, AceTAG-3 and AceTAG-4 and positive control SAHA. AceTAG-1 through AceTAG-4 are depicted in FIGS. 2A and 2B.

FIGS. 9A, 9B, 9C, 9D, 9E and 9F show that AceTAG molecules engage FKBP12^{F36V} and p300/CBP to induce acetylation of target protein. FIG. 9A shows that H3.3-FKBP12^{F36V} chimera localizes primary to chromatin along with endogenous H3.3. FIG. 9B shows that AceTAG-1 inducing modestly higher levels of acetylation relative to the other analogs. FIG. 9C shows no substantial changes in endogenous histone lysine acetylation upon AceTAG treatment, including H3.3, likely due to the substantially lower H3.3-FKBP12^{F36V} acetylation and expression levels (˜25-fold) relative to endogenous histones. FIG. 9D shows that treatment of FKBP12^{F36V}-RelA expressing cells with AceTAG-1 resulted in dose-dependent acetylation at K310, with minimal effects on neighboring K314/315. FIG. 9E shows that a significant level of p53 acetylation is detectable after ˜10 min of AceTAG compound treatment. FIG. 9E that AceTAG-1-induced acetylation is not dependent upon the positioning of the FKBP12^{F36V} tag, as similar acetylation effects were observed with C-terminally tagged p53.

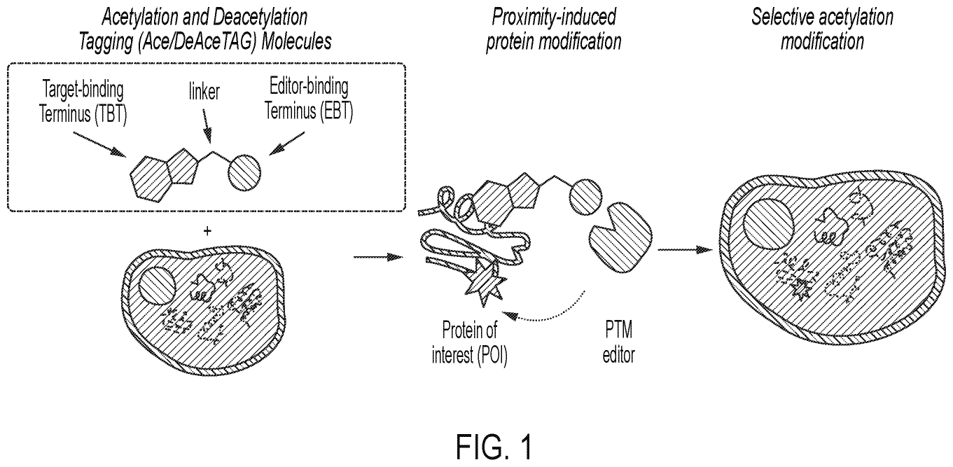

### DETAILED DESCRIPTION

The present invention is directed to embodiments of (De)AceTAG molecules which are composed of a protein-binding terminus (PBT and also termed TBT or target-binding terminus herein) and recruiter-binding terminus (RBT, also termed EBT or editor-binding terminus herein) connected by a chemical linker. The PBT is composed of a small molecule that can engage a protein of interest (POI) and the RBT is composed of a small molecule that engages an enzyme that can either add (e.g. KATs) or remove (e.g. KDACs) acetylation on lysine side chains, resulting in proximity induced modification of the POI.

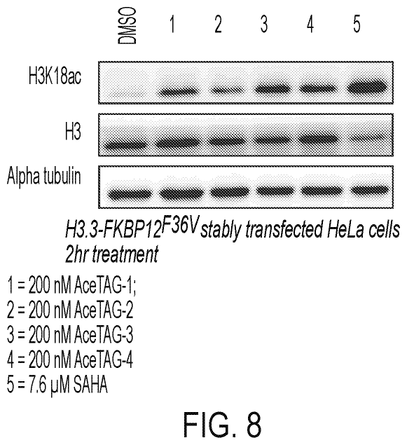

Exemplary embodiments of the AceTAG version of Formula I include two moieties of a recruiter-binding terminus for the P300/CBP or PCAF/GCN5 enzyme and a protein binding terminus for the FKBP12^{F36V} protein and several versions of the linker L as shown in the following formulas, AceTAG-polyol A, AceTAG-polyol B, AceTAG-polyol C, AceTAG-polyol D, AceTAG-polyol E, AceTAG-alkyl A, AceTAG-alkyl B, AceTAG-alkyl C, AceTAG-alkyl D, AceTAG-alkyl E. AceTAG-polyol A with n=1 or 2 (AceTAG-1 and AceTAG-2 respectively and AceTAG-alkyl A with n=4 or 6 (AceTAG-3 and AceTAB-4 respectively) demonstrate significant acetylation of H3.3 as shown in FIG. 8.

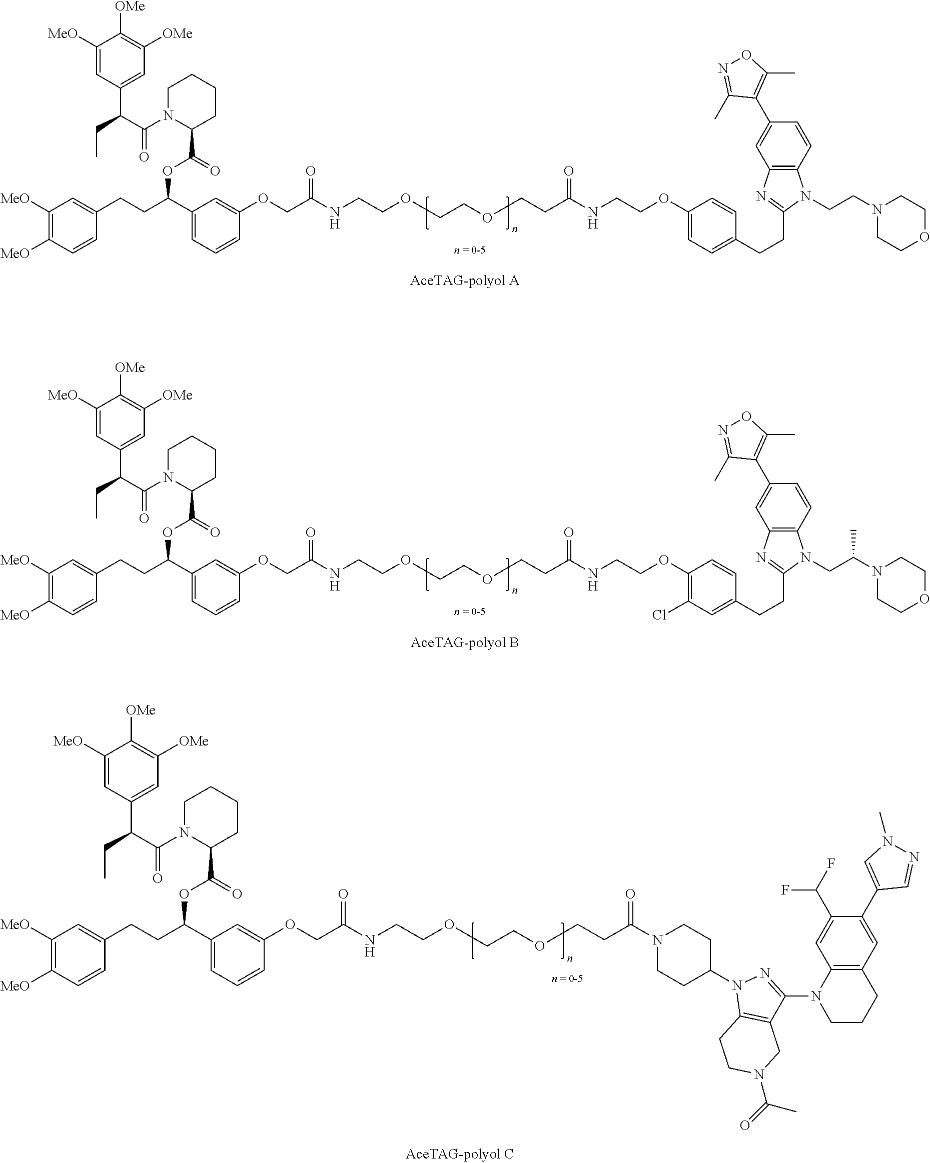

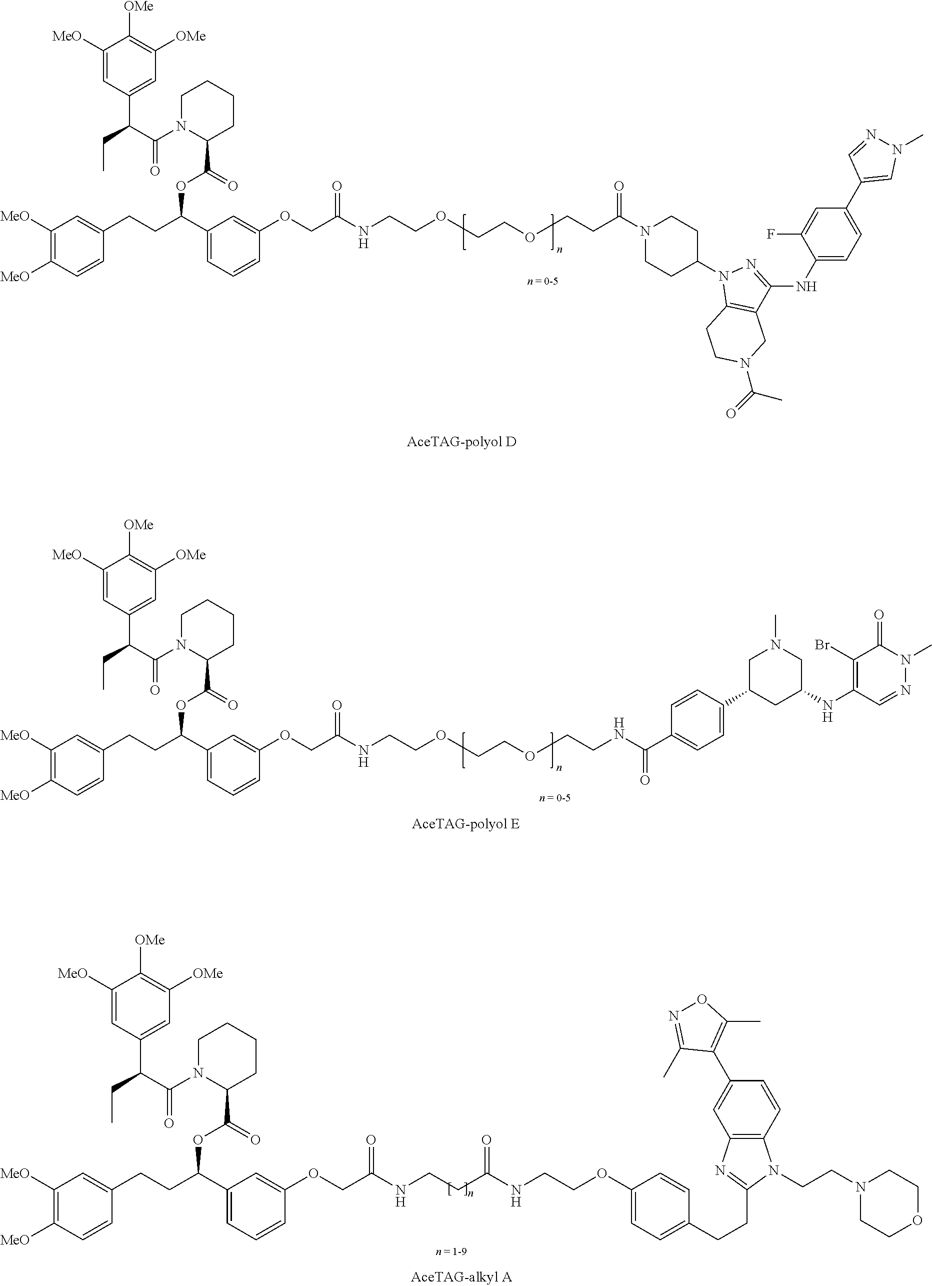

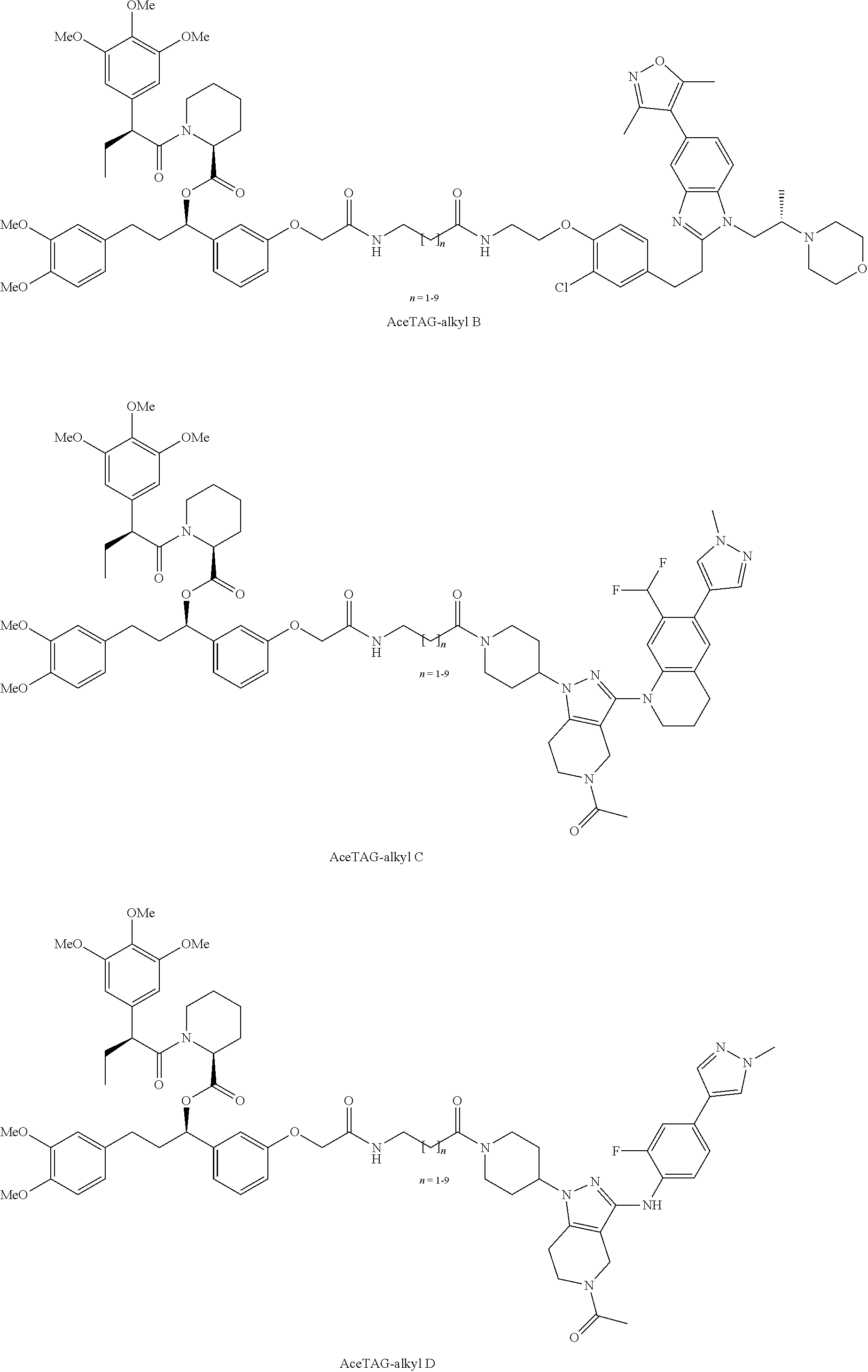

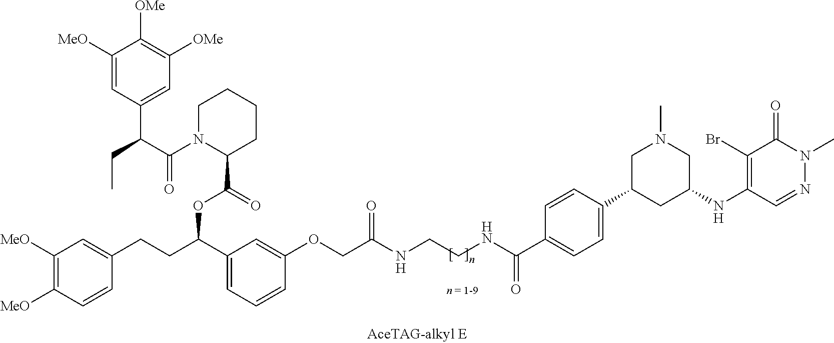

Exemplary embodiments of a DeAceTAG version of Formula I includes the same protein binding terminus for the FKBP12^{F36V} protein and a pair of recruiter binding termini for the SIRT1 and SIRT6 enzymes which deacetylate an acetylated FKBP12^{F36V} protein. The first pair of DeAceTAG embodiments bind with the SIRT1 enzyme. The second pair of DeAceTAG embodiments bind with the SIRT6 enzyme. These embodiments are shown in the following DeAceTAG structures.

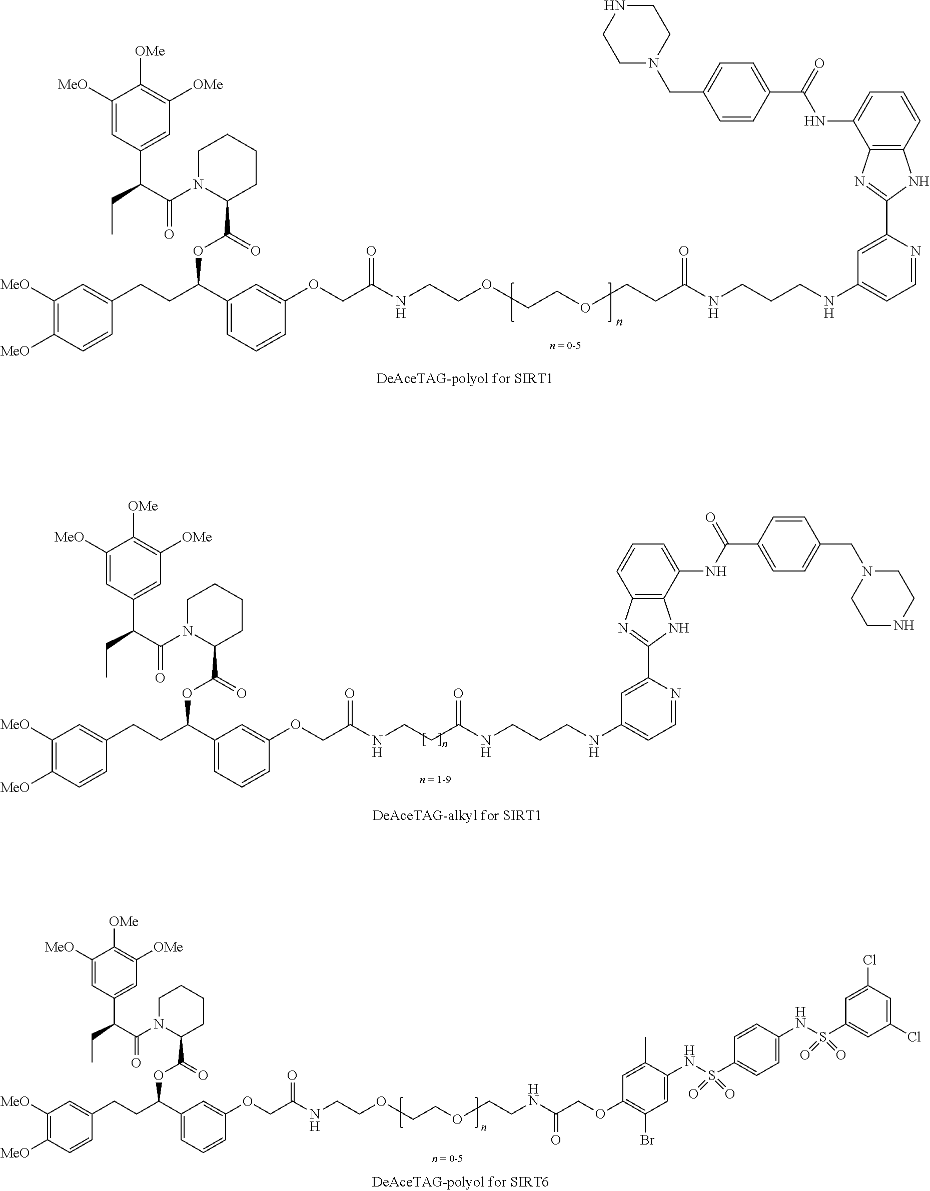

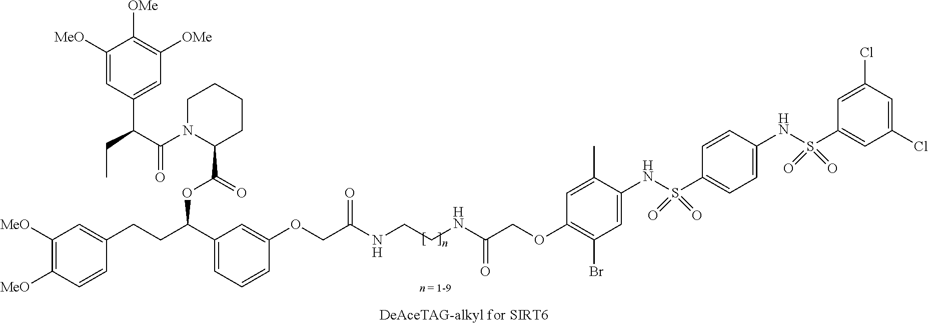

Additional PBT molecules which are useful for probing for certain classes of endogenous proteins include the scout fragments of Formulas S1, S2, S3 and S4, and experimentally confirmed ligands S5 and S6. These scout fragments and ligands may be incorporated into the formulas for AceTAG-polyol A-E and AceTAG-alkyl A-E as replacements of the PBT of these AceTAGs. The scout fragments are capable of selecting classes of endogenous proteins from a gross mixture. Confirmed ligand S5 targets Estrogen Receptors (ER) and S6 targets B-cell lymphoma protein 6 (Bcl6) in context of cellular protein mixtures.

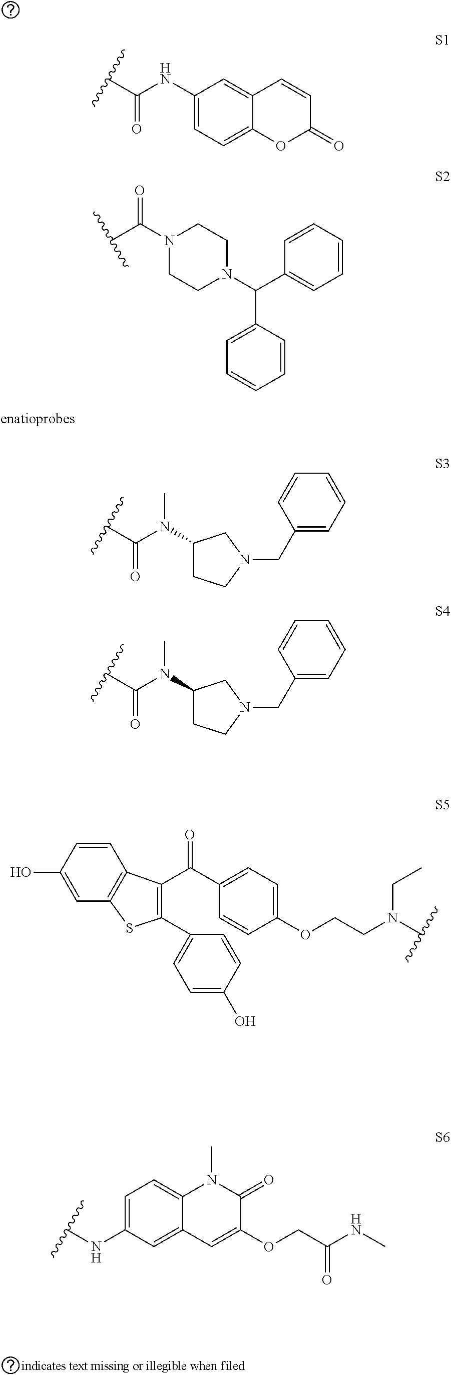 [SMILES: *C(=O)Nc1ccc2oc(=O)ccc2c1 ; *C(=O)N1CCN(C(c2ccccc2)c2ccccc2)CC1 ; *C(=O)N(C)[C@H]1CCN(Cc2ccccc2)C1 ; *C(=O)N(C)[C@@H]1CCN(Cc2ccccc2)C1 ; *N(CC)CCOc1ccc(C(=O)c2c(-c3ccc(O)cc3)sc3cc(O)ccc23)cc1 ; *Nc1ccc2c(c1)cc(OCC(=O)NC)c(=O)n2C]

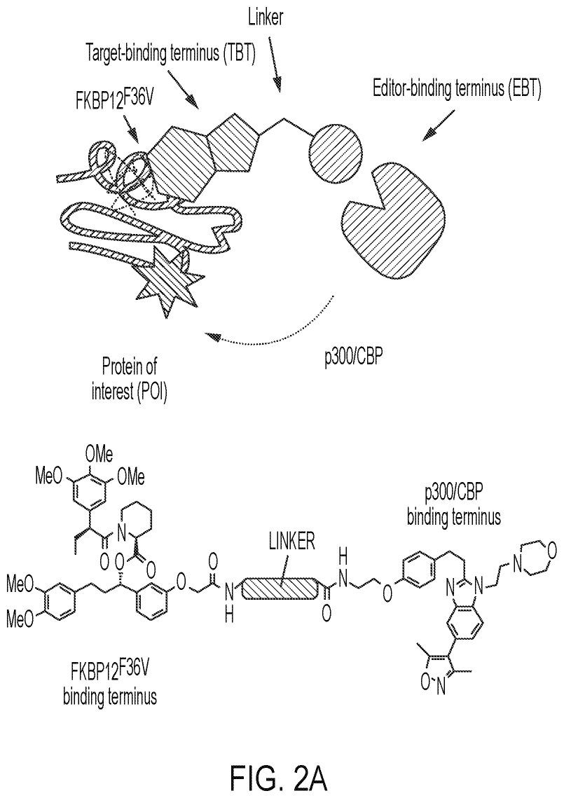

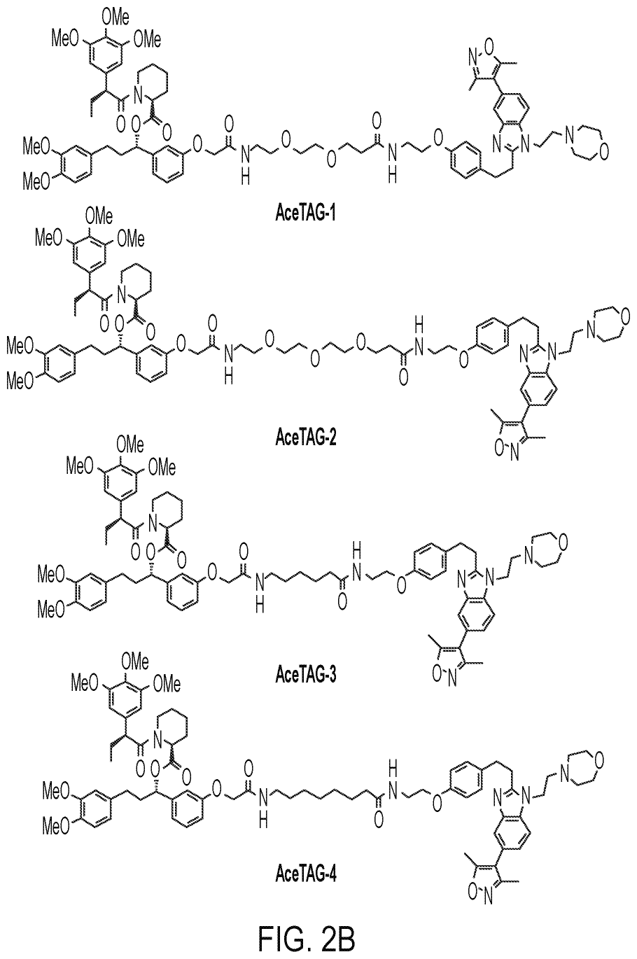

The facility and activity of the PBT-L-RBT molecule is demonstrated by a series of bifunctional molecules (FIGS. 2A and 2B) where a ligand (RBT) for the KAT p300/CBP ^{[6]} is conjugated through a linker L to a ligand (PBT) for the FKBP12^{F36V} protein (AP1867, 14). The enzyme p300/CBP was selected for its broad substrate scope, as it modifies over two-thirds of known acetylation sites ^{[7]}, and the FKBP12^{F36V} protein was used as the PCT to enable exploration of the effects of induced acetylation on proteins without selective small molecule ligands. Pursuant to this design strategy, a small library of AceTAG molecules were synthesized by combining the p300/CBP bromodomain ligand and FKBP12^{F36V} ligand through linkers varying in length and composition (AceTAG-1-4, FIG. 2B).

### AceTAG Molecules Engage FKBP12^{F36V} and p300/CBP to Induce Acetylation of Target Protein

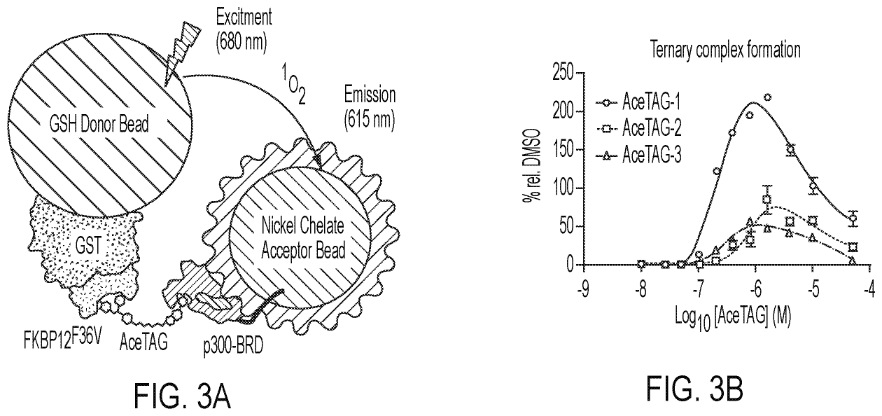

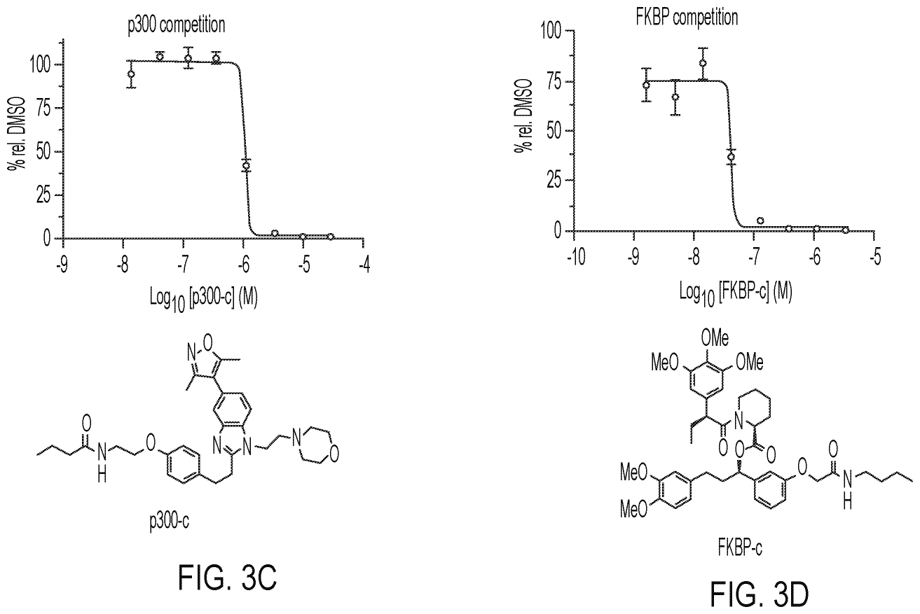

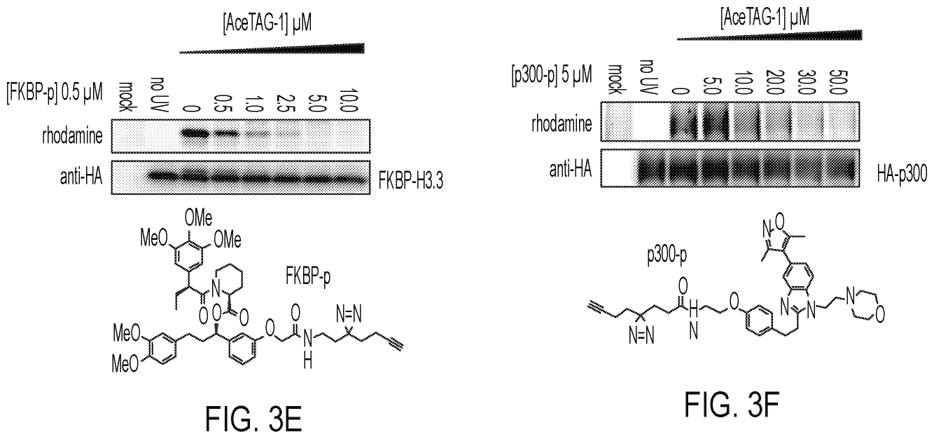

To access the ability of AceTAG molecules to mediate complex formation between soluble recombinant FKBP12^{F36V} and the BRD domain of p300 (BRD-p300) in vitro using an AlphaScreen assay (Winter et al., Science 2015, 348:1376-1381) (FIG. 3A). We observed characteristic bell-shaped autoinhibitory curves for all AceTAG analogues, the result of AceTAG molecules saturating both FKBP12^{F36V} and BRD-p300, effectively outcompeting ternary complex formation. We noted that AceTAG-1 has increased potence relative to other analogues, with maximum complex formation occurring at ˜1 μM (FIG. 3B). In addition, the luminescence signal is considerably diminished upon co-treatment of either terminal binding molecules in a concentration-dependent fashion (FIGS. 3C and 3D), confirming that observed ternary complex formation is dependent on the simultaneous binding of both BRD-p300 and FKBP12^{F36V} To verify that AceTAG molecules can engage their protein binding partners in cells, we constructed “fully functionalized” photoaffinity probes of both the FKBP12^{F36V} (FKBP-p) and p300/CBP (p300-p) ligands and confirmed respective binding to recombinantly expressed p300 and FKBP12^{F36V} can be efficiently competed when cells are co-treated with increasing concentrations of AceTAG-1 (FIGS. 3E and 3F). Together, these biochemical data indicate that AceTAG molecules effectively engage FKBP12^{F36V} and p300/CBP and are capable of inducing complex formation.

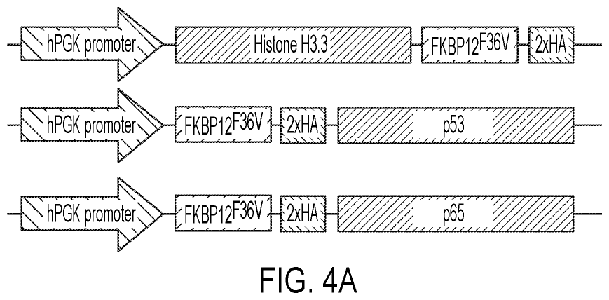

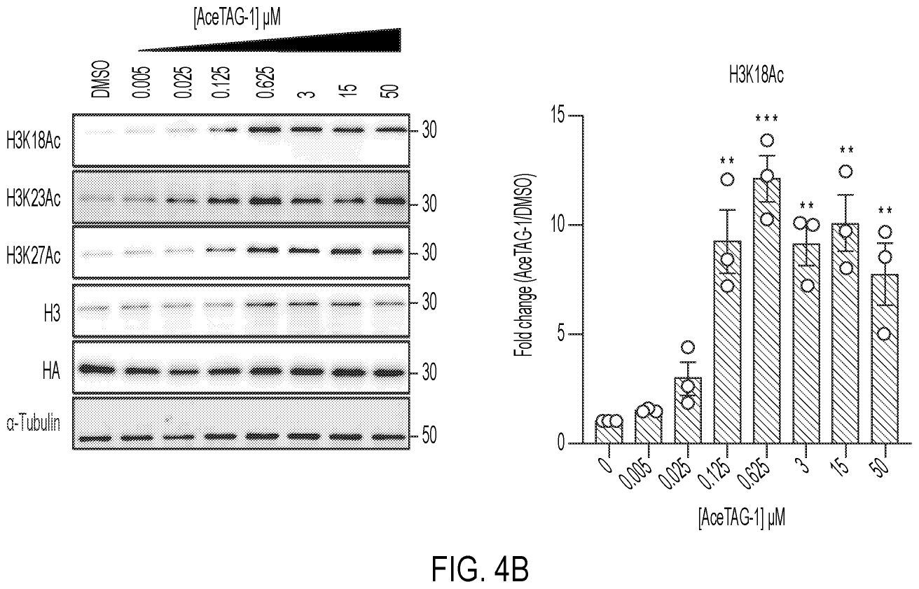

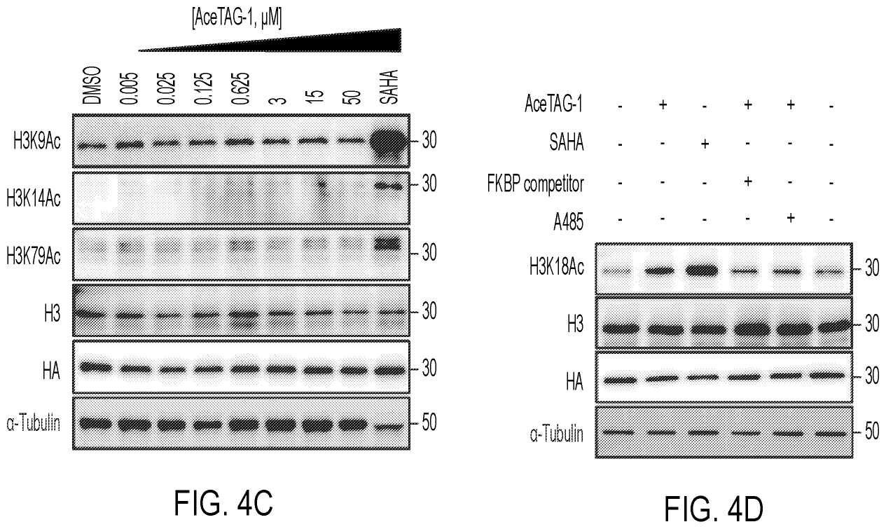

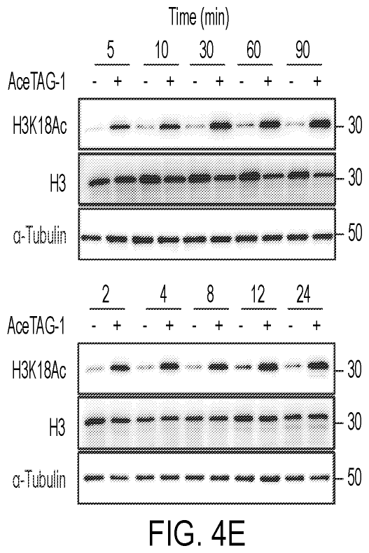

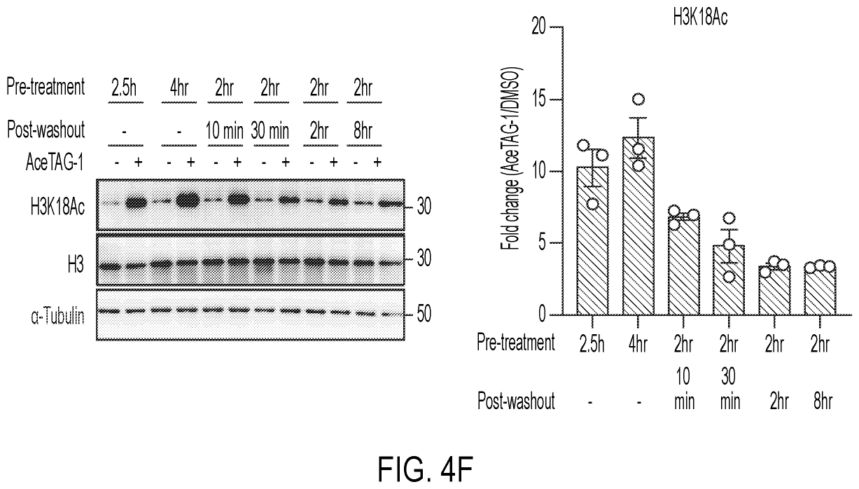

The bio-ability of the ternary complex PBT-L-RBT as described above for induction of acetylation on protein of interest, was demonstrated in the context of bioengineered use of generated cell lines stably expressing FKBP12^{F36V} fusion proteins of Histone H3.3, p53, and RelA (FIG. 4A) which are three known p300/CBP substrates ^{[8, 30, 31]}. Acetylation is a critical regulator for these protein function but it mediates distinct cell signaling pathways on different proteins. Acetylation on H3.3 loosens compact chromatin and activate genome-wide transcription ^{[9]}, while acetylation of p53 and RelA modulate their transcriptional activity. Despite unanimous importance of acetylation on these proteins, the function of site-specific acetylation remains uncertain due to limited tools.

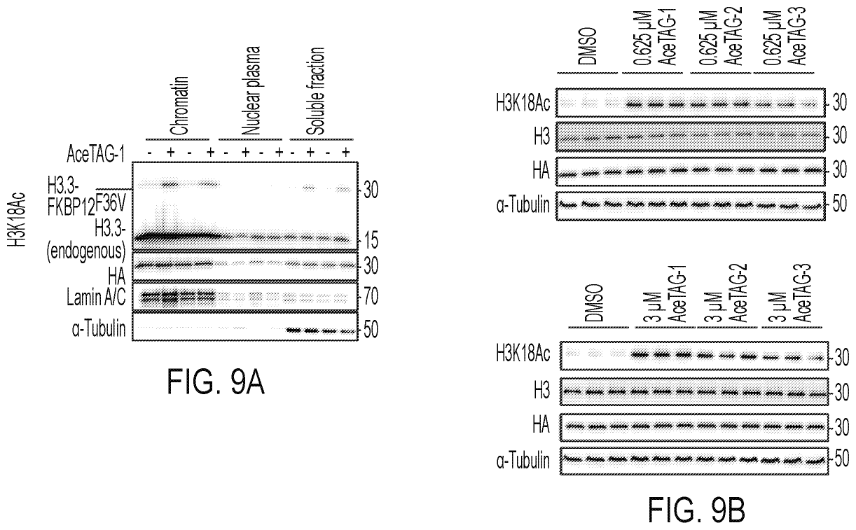

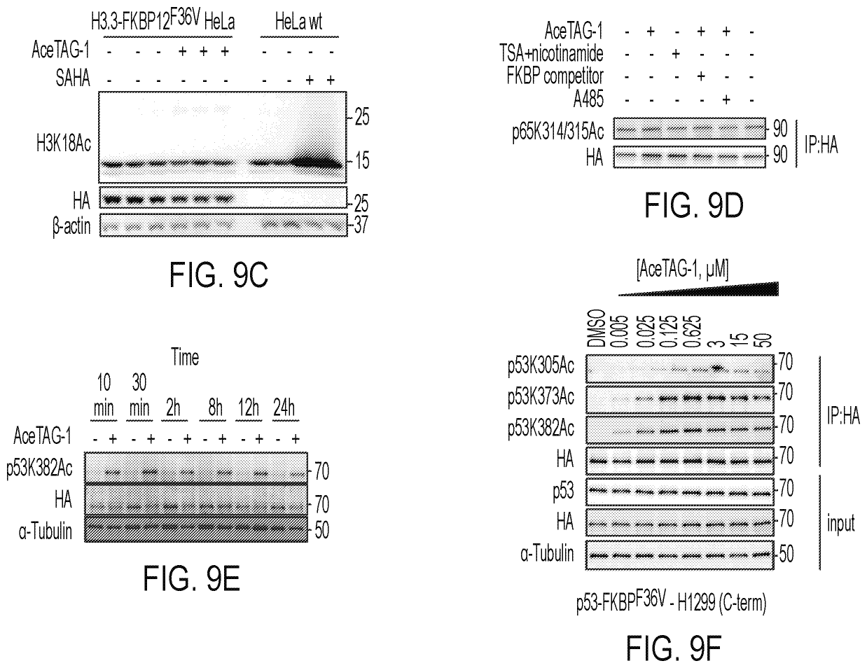

To evaluate the properties of AceTAG molecules, we first stably transfected HeLa cells with H3.3-FKBP12^{F36V}-HA and confirmed that the H3.3-FKBP12^{F36V} chimera localizes primary to chromatin along with endogenous H3.3 (FIG. 9A). The H3.3-FKBP12^{F36V} HeLa cell line was treated with increasing concentrations of AceTAG-1 and a dose-dependent increase of H3.3 acetylation at lysine 18, 23, and 27 but not lysine 9, 14, or 79 was observed (FIGS. 4B and FIG. 4C). This result matched the recent published evidence that p300/CBP does not alter acetylation level changes of H3K9 and H3K14 ^{[5]}. We observed AceTAG-mediated K18 acetylation for all analogs, with AceTAG-1 inducing modestly higher levels of acetylation relative to the other analogs (FIG. 9B). As expected, loss of acetylation level was observed at 50 μM AceTAG-1 treatment, likely due to autoinhibition, or the ‘hook effect’, often observed in ternary complexes ^{[10]}. The induction of acetylation of H3.3-FKBP12^{F36V} in a p300/CBP-dependent manner by AceTAG-1 was also demonstrated. This induction was demonstrated by showing that acetylation of H3.3-FKBP12^{F36V} was blocked with treatment of excess FKBP12^{F36V} ligand and p300/CBP catalytic inhibitor, A485 (FIG. 4D).

To obtain a better idea of AceTAG kinetics, H3.3-FKBP12^{F36V} HeLa cell line was treated with 625 nM AceTAG-1 over increasing periods of time. Surprisingly, acetylation level of the H3.3-FKBP12^{F36V} fusion significantly increased only after a 5-minute treatment (FIG. 4E), indicating a rapid induction by AceTAG-mediated p300/CBP recruitment. In addition, when AceTAG-1 is removed from cells, H3.3 K18 acetylation steadily dissipates, reaching near basal levels within 2 hr after compound removal (FIG. 4F). This re-equilibration is likely the result of endogenous KDAC activity, together indicating that the targeted acetylation is reversible and dependent on the heterobifunctional compound. This highlights AceTAG's potential to be an effective chemical tool to study the immediate and primary effects of protein acetylation.

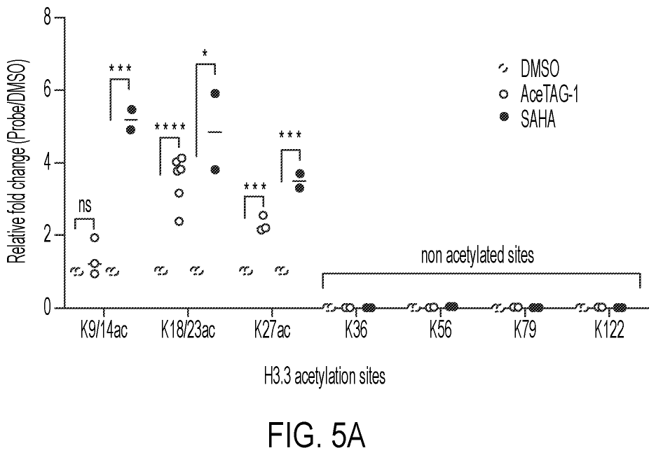

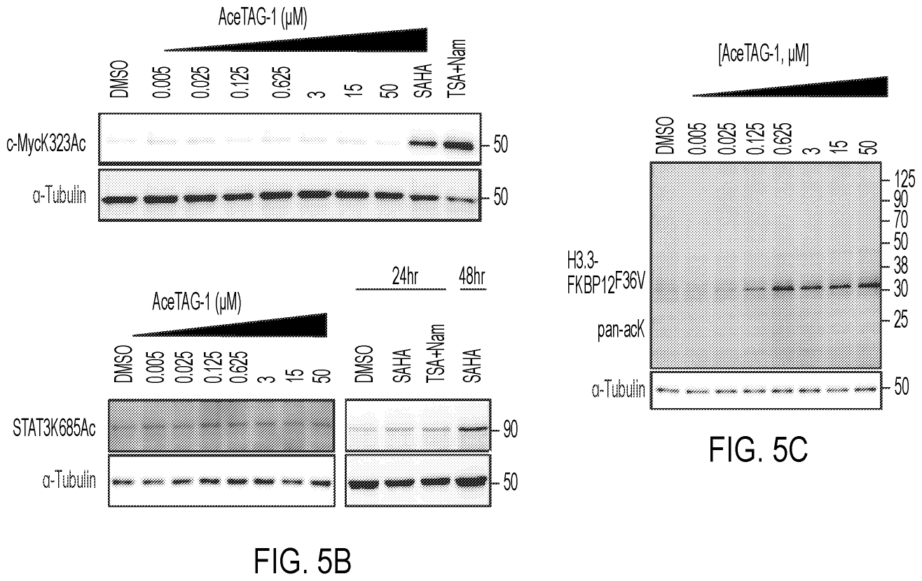

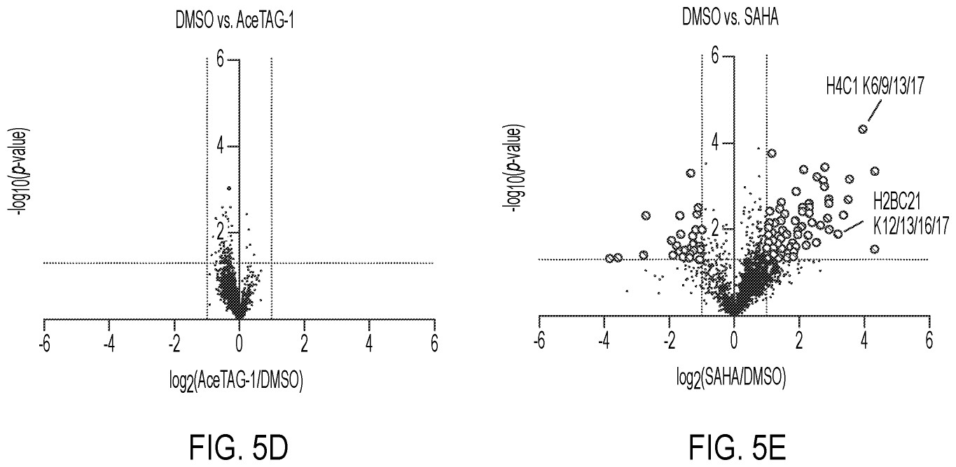

To more robustly characterize the site selectivity of induced acetylation of targeted proteins using AceTAG, we monitored AceTAG-1-induced acetylation in cells by quantitative mass spectrometry (MS). In these experiments, H3.3-FKBP12^{F36V}-HA was enriched from stably transfected HeLa cells treated with DMSO, AceTAG-1, or SAHA and trypsinized for protein identification and quantitation by using tandem mass tags (TMT). In line with immunoblotting experiments (FIGS. 4B and 4C), we measured substantial increases in acetylation at K18, K23, and K27 but detected no acetylation at other identified lysines on H3.3 (FIG. 5A). Together, these data suggest that when p300/CBP is recruited to H3.3 in cells by using our AceTAG system, the preferred acetylation sites are K18, K23, and K27.

We next sought to evaluate the selectivity of AceTAG-induced acetylation across the human proteome. Treatment of HeLa cells with AceTAG-1 resulted in no observable acetylation changes in well-established p300/CBP substrates, including c-Myc and STAT3 (FIG. 5B) or broader acetylation perturbation via immunoblot assays (FIG. 5C). To assess targeted acetylation selectivity more globally and quantitatively, we performed MS-based acetylproteomic analysis of AceTAG-treated H3.3-FKBP12^{F36V} HeLa cells. Briefly, acetylated peptides from H3.3-FKBP12^{F36V} HeLa cells treated either with DMSO, AceTAG-1, or SAHA were digested, enriched, identified, and quantified by using TMT (FIG. 5D). Relative to DMSO, we observed no substantial changes in acetylation of any detected protein in AceTAG-treated cells while SAHA treatment resulted in increases across ˜30 proteins (FIG. 5E). We note that no substantial changes in endogenous histone lysine acetylation was observed upon AceTAG treatment, including H3.3, likely due to the substantially lower H3.3-FKBP12^{F36V} acetylation and expression levels (˜25-fold) relative to endogenous histones (FIG. 9C). Collectively, these data suggest that AceTAG-1 does not broadly affect p300/CBP KAT activity on other substrates, and AceTAG-1 mediated acetylation is endowed with exquisite selectivity for FKBP12^{F36V}-tagged proteins.

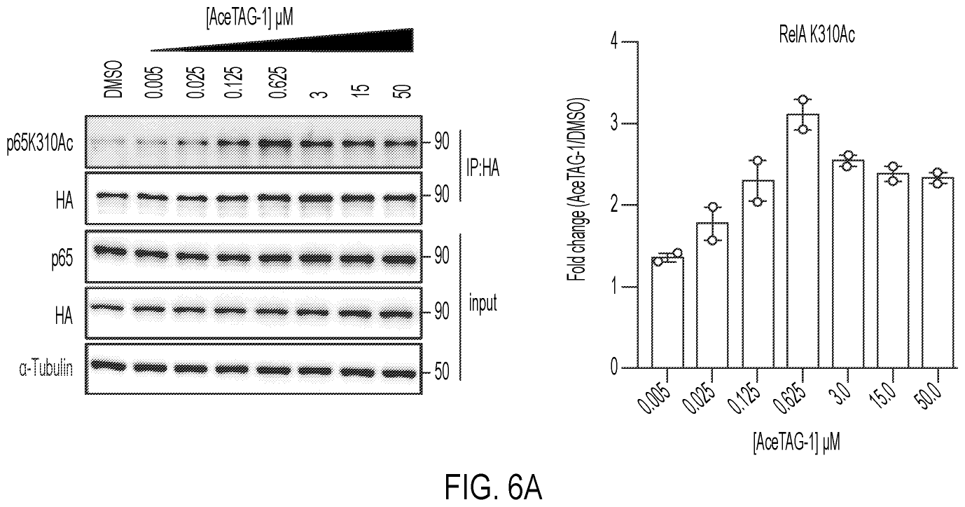

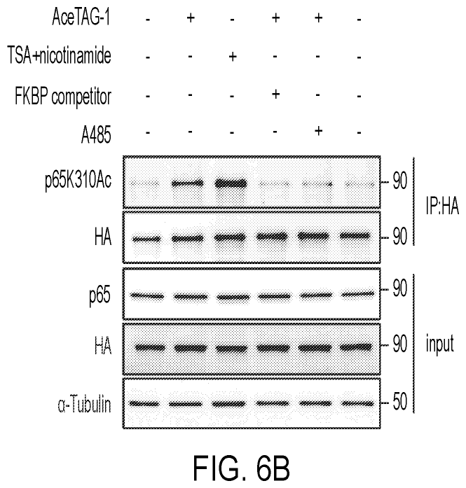

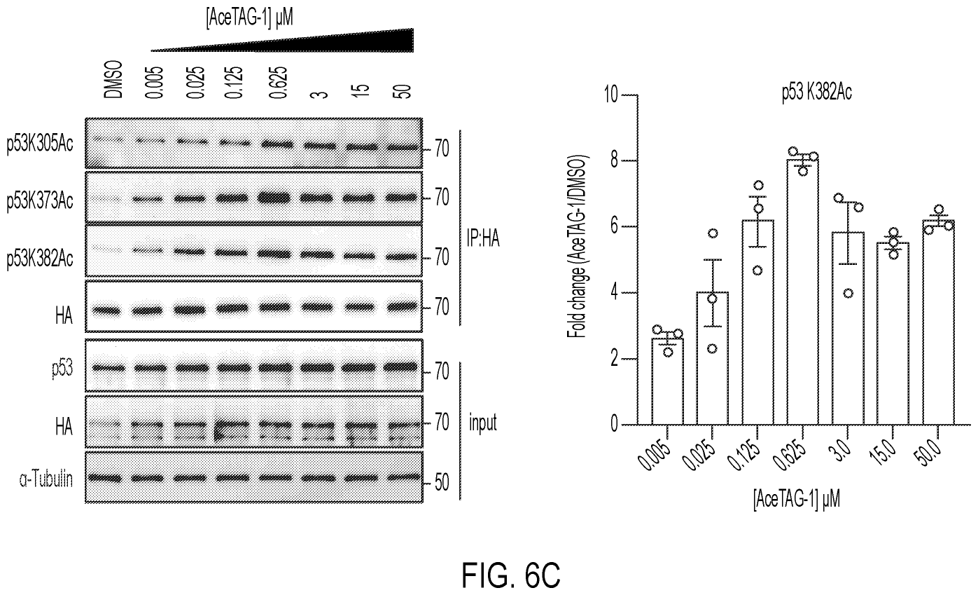

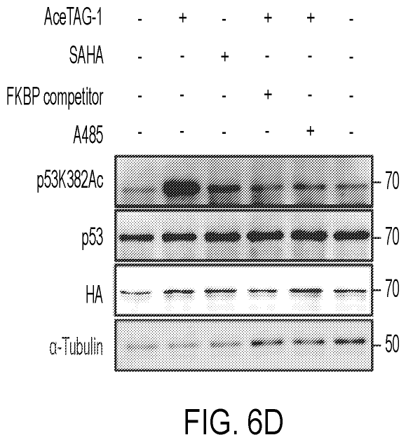

In addition to H3.3-FKBP12^{F36V} fusion protein, the acetylation of FKBP12^{F36V}-RelA and FKBP12^{F36V}-p53 fusion protein by AceTAG-1 were examined. To avoid potential obfuscation by endogenous target proteins, FKBP12^{F36V}-RelA was stably transfected in a HeLa RelA−/− cell line while FKBP12^{F36V}-p53 was stably transfected in H1299 non-small cell carcinoma (NSCLC) cells, which have a homozygous partial deletion of TP53 and lack p53 protein expression. Treatment of FKBP12^{F36V}-RelA expressing cells with AceTAG-1 resulted in dose-dependent acetylation at K310, a site previously proposed to be a predominant target of p300/CBP (Chen et al., Mol. Cell. Biol. 2005, 25:7966-7975) (FIG. 6A) with minimal effects on neighboring K314/315 (FIG. 9D). In FKBP12^{F36V}-p53 H1299 cells, we monitored acetylation of the C-terminal domain of p53, specifically at K305, K373, and K382, sites previously suggested to be substrates of p300/CBP where strong AceTAG-1-dependent acetylation was also observed (FIG. 6C). Consistent with our previous observations, we observe apparent acetylation autoinhibition for both targets at higher AceTAG-1 concentrations (FIGS. 6A and 6C) and blockade of induced acetylation upon co-treatment of cells with p300/CBP KAT inhibitor A-485 or competing FKBP ligand (FIGS. 6B and 6D). In addition, a significant level of p53 acetylation is detectable after ˜10 min of AceTAG compound treatment (FIG. 9E), similar to the kinetics observed for H3.3. Finally, we note that AceTAG-1-induced acetylation does not appear dependent upon the positioning of the FKBP12^{F36V} tag, as similar acetylation effects were observed with C-terminally tagged p53 (FIG. 9F).

Histone 3.3 (H3.3) is deposited on chromatin independent of replication, and is predominantly found in the gene body of actively transcribed genes at transcription start sites (TSS), and in regulatory regions ^{[20]}. Extensive studies have established that the acetylation of histone H3.3 by p300/CBP is primarily associated with gene activation ^{[21]}. However, these

studies have relied on either depletion or inhibition of p300/CBP, ^{[22]} which only allow insight into the functional consequences induced by loss of basal acetylation at gene loci and can result in confounded interpretation due to the broad substrate scope of p300/CBP. Thus, a complete understanding of histone acetylation requires illumination of the p300/CBP mediated acetylation enhancement from basal level.

The present invention advances this understanding through the utilization of stable expression or knock-in FKBP12^{F36V}-H3.3 fusions. The use of AceTAG molecules with these fusions to induce histone acetylation enables the examination of the kinetic aspects of concomitant functional changes regarding transcriptome-wide regulation ^{[23]}, telomere stability ^{[24]}, and heterochromatin maintenance ^{[25]}.

### Investigation of p53 Acetylation Using AceTAG Systems

The AceTAG system was also employed to investigate consequences of acetylation in human cells. Inactive p53, either non-acetylated or mutant p53, is the most frequent tumor

suppressors in human cancers ^{[12]}. Acetylation of p53 by p300/CBP is critical for maintaining its stability, increasing transcription activity of apoptotic gene targets, and mediating checkpoint responses to DNA damage ^{[8a, 13]}. Most of these studies relied on mutations of lysine to arginine or glutamine to block or mimic acetylation, which does not fully recapitulate acetylated p53 and protein dynamics. Furthermore, it has yet been firmly established as to what specific sites, when acetylated, predominantly control p53 activity, such as transcriptional activation.

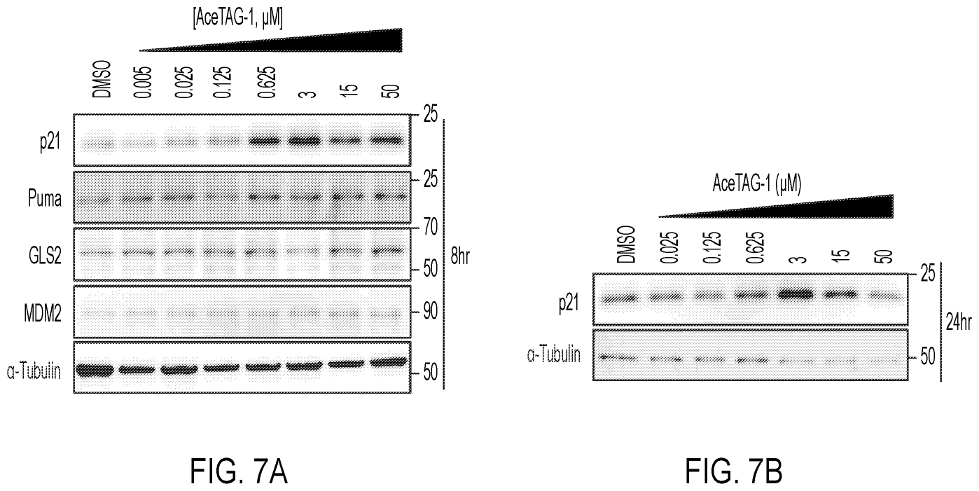

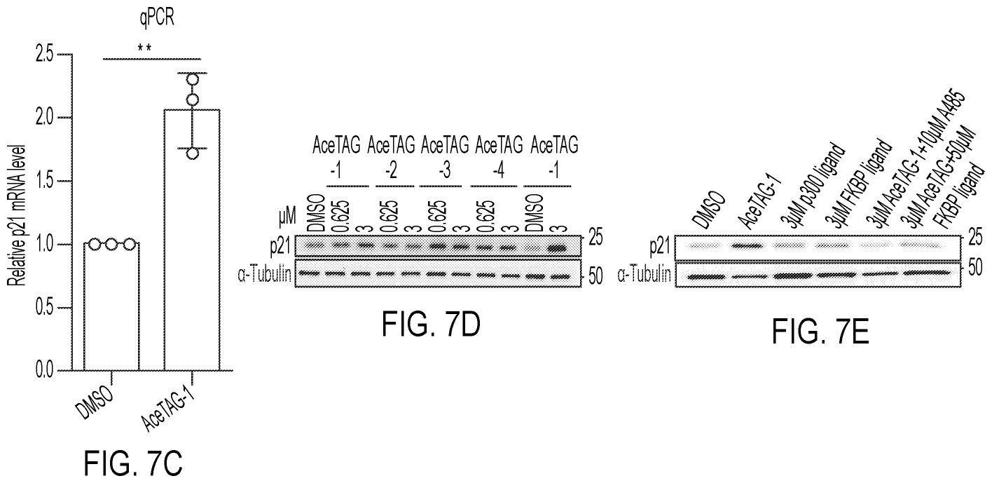

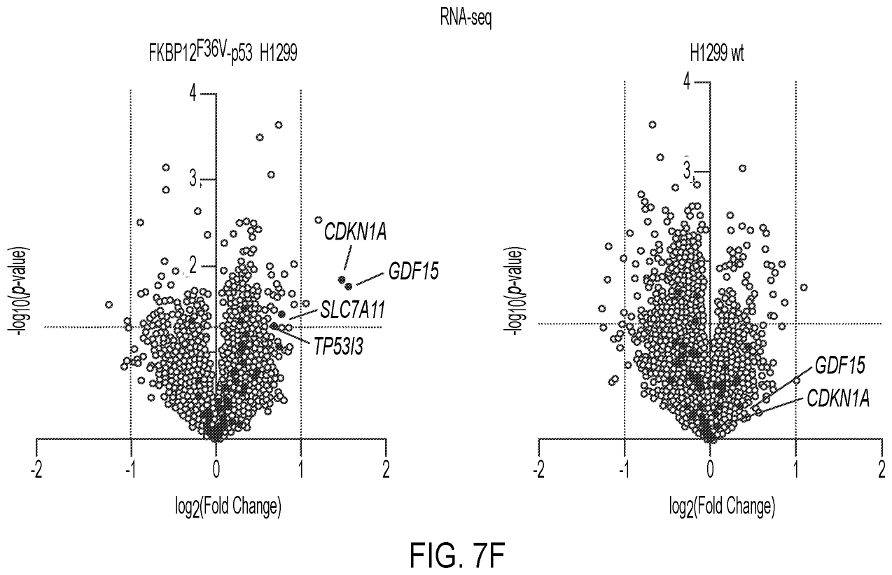

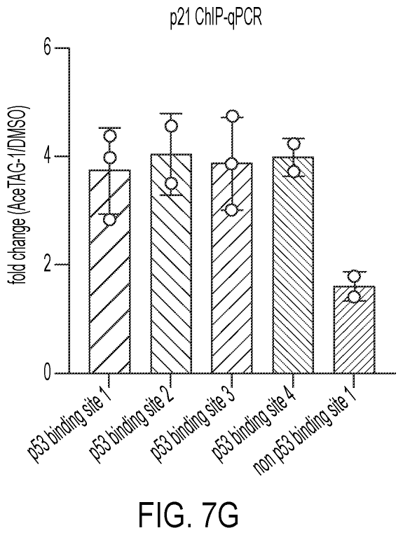

The AceTAG system was applied to study the effects of p53 acetylation in H1299 cell line expressing FKBP12^{F36V}-p53 fusion protein. We first examined if treatment with AceTAG-1 affected genes previously reported to be dependent on p300/CBP-mediated p53 acetylation, which include p21 (CDKNIA), PUMA, MDM2, and GLS2 (Fischer, Oncogene 36, 3943-3956, 2017). Interestingly, AceTAG-based recruitment of p300/CBP to FKBP12^{F36V}-p53 and subsequent acetylation lead to insignificant effects on these target genes except for p21 (FIG. 7A), where AceTAG-1 induces strong dose-dependent expression, observable after 8 hr and 24 hr exposure (FIGS. 7A and 7B). Profoundly, p21 expression level also reflected the ‘hook effect’ of higher concentration AceTAG-1 treatment (FIGS. 7A and 7B). Transcription of p21 was also confirmed by qPCR analysis (FIG. 7C). More importantly, these effects were not observed in H1299 cell line expressing p53 fusion protein without the FKBP12^{F36V} tag (FIG. 7D). The p53 expressing H1299 cells were tested with four different AceTAG molecules and no p21 induction were observed as predicted, which further supports that these effects are dependent on recruitment of AceTAG molecules to FKBP12^{F36V} fusion protein.

To further confirm that AceTAG-mediated p21 activation is dependent on binding of AceTAG to both FKBP12^{F36V}-p53 and p300/CBP, the cells were treated with 3 μM p300 ligand (p300-c) or 3 μM FKBP ligand (FKBP-c) (FIG. 7E). As expected, neither p300 nor FKBP ligand alone increases p21 expression level comparable to AceTAG-1. Moreover, p21 expression was blocked when the cells were incubated with AceTAG-1 and excess FKBP12^{F36V} ligand or p300/CBP catalytic inhibitor, A485.

To examine the effects of AceTAG-1 on p53 more broadly, we performed RNA-sequencing to quantify the transcriptional changes upon p300/CBP recruitment and acetylation. Compared to vehicle, treatment of FKBP12^{F36V}-p53 H1299 cells with AceTAG-1 led to significant upregulation of several transcripts, with the highest two being the TP53 target genes GDF15 (˜3-fold) and CDKNIA (˜2.8-fold), and to a lesser extent, SLC7A11 (˜1.7 fold) and TP5313 (˜1.6 fold) (FIG. 7F). Notably, we observe minimal changes of TP53 target genes in AceTAG-1 treated H1299 wt cells. Though gene set enrichment analysis (GSEA) revealed on-pathway enrichment of TP53 genes, intriguingly we observed little to no increases in targets that affect apoptotic (e.g. Bax, Puma). metabolic (GLS2) or autoregulatory (Mdm2) functions of p53 activation, consistent with immunoblot experiments (FIG. 7A), together suggesting that AceTAG recruitment of p300/CBP to p53 mainly results in transcriptional activation of CDKNIA.

The p53/p21 ^{CIP/WAF1} pathway is one of the most well-studied pathways related to p53 acetylation ^{[8a, 14]}. It has been reported that acetylation of p53 at lysine 373 and 382 play an important in activating transcription and translation of the p21^{CIP/WAF1} gene^{[15]}, which inhibits cell cycle progression during G1 and G2 phases ^{[16]}. However, it is still unknown whether these are the only two sites necessary for activation of downstream pathway. Moreover, the molecular mechanism as to how AceTAG-1 mediates p21 induction is currently uncertain as there are multiple hypotheses of how p53 acetylation affects transcriptional activity. Several studies suggest that acetylation of p53 by p300/CBP increases sequence-specific DNA binding to targeted promoter regions resulting in increased gene activation^{[8, 11]} while others conclude that p53 acetylation can recruit coactivators to target promoters, resulting in neighboring histone acetylation thereby activating target gene transcription (Ho et al., Mol Cell Biol 25, 7423-7431, 2005). We first tested whether AceTAG-mediated p53 acetylation increases its DNA binding to p21 promoter by ChIP-qPCR. The cells were incubated with AceTAG-1 and FKBP12^{F36V}-HA-p53 was immunoprecipitated, where DNA binding to the protein was purified for qPCR analysis. Among five p21 DNA sequences, binding of FKBP12^{F36V}-p53 to four previously reported p53 binding sites on p21 promoter (Mujtaba et al., Molecular Cell 13, 251-263, 2004) were increased with AceTAG-1 treatment. However, no significant increase was observed at the non p53 binding sites within the C-terminus of p21 gene (FIG. 7G). This indicated that AceTAG-mediated p53 acetylation induce p21 activation likely through increasing binding of p53 to p21 promoter but further experiments are needed to determine detailed mechanism.

### Cancer Treatment Using AceTAG systems

The p53 system is the most frequently inactivated tumor suppressor system in human cancers, particularly in non-small cell lung carcinomas (NSCLC). To date, no successful therapies have been developed against it ^{[17]}. Acetylation of p53 by p300/CBP is critical for its tumor suppressive functions by increasing stability, binding to low affinity promoters, association with other proteins, and it is required for its checkpoint responses to DNA damage and activated oncogenes ^{[8a, 18]}. However, most of these studies rely on mutations of lysine to arginine or glutamine [^{8a, 18b, 18c, 18e, 18f]}, which do not fully mimic lysine acetylation functions nor recapitulate the complex dynamics carried out by the interplay of KATs and KDACs. Thus, the understanding of the biological consequences of p53 acetylation by p300/CBP in cancer cells remains incomplete ^{[19]}.

Pursuant to embodiments and aspects of the present invention, the AceTAG can be used with stable expression and knock-in systems to induce acetyl-p53 in situ (NSCLC cell lines). These aspects will enable examination of the effects on p53 interactions (e.g. Mdm2), activation, and stability as well as cell growth arrest, apoptosis, and cellular stress.

The proto-oncogene BCL6 is a transcriptional repressor necessary for germinal-center formation and has been implicated in the pathogenesis of B-cell lymphomas ^{[26]}. Recent evidence suggests that p300-mediated acetylation of BCL6 negatively regulates transcriptional repression and pharmacological inhibition of KDACs leads to accumulation of inactive acetylated BCL6 in B-cell lymphoma cells, implying that enhancing BCL6 acetylation may be a promising therapeutic strategy for lymphoma ^{[27]}. Most studies have relied on inducing BCL6 acetylation indirectly through broad blockade of KDACs using histone deacetylase inhibitors (HDACi). According to the present invention, the AceTAG system can be employed to explore the effects of specific induction of BCL6 acetylation on its transcriptional activity, cell cycle, proliferation and apoptosis in B-cell lymphoma lines.

Estrogen receptors (ER) are a group of nuclear receptors activated through interaction with estrogens. Among them, ER α and ER β have been extensively studied for their roles in multiple cellular signaling pathways and diseases. A group of drugs called SERMs (selective estrogen receptor modulators) target ER α or ER β to treat various estrogen-related diseases including ovulatory dysfunction, postmenopausal osteoporosis, breast cancer and genitourinary syndrome of menopause. However, their molecular functions are complicated not only by different selectivities between α and β form, but also by different co-activator or co-inactivator proteins that are preferentially recruited after binding with SERMs.

According to the present invention, hetero-bifunctional moieties comprising PBTs targeting ER and RBTs targeting acetylase enzymes could exert definitive and predicable effects on ER downstream genes by recruiting either transcription activators or deactivators, therefor hold potential as a platform to customize novel ER modulators.

### Methods and Materials

Materials: Mouse Anti-HA (2367S, 1:2000 dilution), rabbit anti-HA (3724S, 1:2000 dilution), rabbit anti-H3K9ac (9649S, 1:2000 dilution), rabbit anti-H3K14ac (7627S, 1:2000 dilution), rabbit anti-H3K18ac (13998S, 1:2000 dilution), rabbit anti-H3K27ac (8173S, 1:2000 dilution), rabbit anti-H3 (4499S, 1:2000 dilution), rabbit anti-acetyl lysine (9814S, 1:1000 dilution), rabbit anti-acetyl-p65 (Lys310) (3045S, 1:1000 dilution), rabbit anti-NFkBp65 (8242S, 1:2000 dilution), rabbit anti-p53K382ac (2525S, 1:1000 dilution), rabbit anti-Stat3K685ac (2523S, 1:2000 dilution), rabbit Lamin A/C antibody (2032S, 1:1000 dilution), and anti-rabbit IgG HRP conjugate (7074P2, 1:10000 dilution) were from Cell Signaling Technology. Anti-tubulin hFAB Rhodamine (12004166, 1:3000 dilution) was from Bio-rad. Rabbit anti-H3K23ac (07-355, 1:2000 dilution), rabbit anti-H3K79 (SAB5600231-100UG, 1:2000 dilution), rabbit anti-c-MycK323ac (ABE26, 1:2000 dilution) were from Millipore Sigma. rabbit anti-p53K373ac (ab62376, 1:1000 dilution), and rabbit anti-p53K305ac (ab109396, 1:1000 dilution) were from Abcam. Mouse anti-p53 (628202, 1:2000 dilution) was from Biolegend. Rabbit anti-p65K314/315ac (PA5-114696, 1:500 dilution), Anti-mouse IgG (H+L) HRP conjugate (PA1-28568, 1:10000 dilution) was from Thermo Fisher Scientific.

Cell Culture: HEK293T and HeLa cells (ATCC) were cultured in DMEM supplemented with 10% FBS, 1% (v/v) penicillin/streptomycin, and 2 mM glutamine. H1299 (ATCC) were cultured in RPMI supplemented with 10% FBS, 1% (v/v) penicillin/streptomycin, 2 mM glutamine, 10 mM HEPES, and 1 mM sodium pyruvate. RelA KO HeLa cells (Abcam) we cultured in DMEM supplemented with 10% FBS, 1% (v/v) penicillin/streptomycin, and 2 mM glutamine. All cells were cultured at 37° C. and 5% CO₂.

Lentivirus Plasmid Construction: pLEX_305-N-dTAG and pLEX305-C-dTAG empty

| Insert | | |
|---|---|---|
| Gene | | |
| (Human) | Forward (5′-3′) | Reverse (5′-3′) |
| Histone | GGGGACAAGTTTGTACAA | GGGGACCACTTTGTACAAG |
| H3.3 (C- | AAAAGCAGGCTTCATGGC | AAAGCTGGGTCAGCACGTT |
| terminus) | TCGTACAAAGCAGACTG | CTCCACGTATGC |
| | (SEQ ID NO: 1) | (SEQ ID NO: 2) |
| p53 | GGGGACAAGTTTGTACAA | GGGGACCACTTTGTACAAG |
| (N- | AAAAGCAGGCTTCGAGGA | AAAGCTGGGTCTCAGTCTG |
| terminus) | GCCGCAGTCAGATCC | AGTCAGGCCCTTC |
| | (SEQ ID NO: 3) | (SEQ ID NO: 4) |
| p53 | GGGGACAAGTTTGTACAA | GGGGACCACTTTGTACAAG |
| (C- | AAAAGCAGGCTTCATGGA | AAAGCTGGGTCGTCTGAGT |
| terminus) | GGAGCCGCAGTCAG | CAGGCCCTTC |
| | (SEQ ID NO: 5) | (SEQ ID NO: 6) |
| p65 | GGGGACAAGTTTGTACAA | GGGGACCACTTTGTACAAG |
| (N- | AAAAGCAGGCTTCGACGA | AAAGCTGGGTCTTAGGAGC |
| terminus) | ACTGTTCCCCCTCATCTT | TGATCTGACTCAGCAGG |
| | C (SEQ ID NO: 7) | (SEQ ID NO: 8) |

vectors were kindly provided by Dr. Erb (The Scripps Research Institute). Gateway recombination cloning technology (Invitrogen) was used to clone targets of interest into pLEX_305-N-dTAG and pLEX_305-C-dTAG. H3.3 cDNA ORF clone in pGEM-T vector was purchased from SinoBiological (Cat# HG16451-G). p53 cDNA ORF clone in pCMV3-C-HA vector was purchased from SinoBiological (Cat# HG10182-CY). RelA cDNA ORF clone was purchased from SinoBiological (Cat# HG12054-G). H3.3, p53, and RelA were cloned into Gateway compatible donor vector pDONR221 using BP clonase after PCR with primers containing BP overhangs sequence from the vector described above. H3.3 and p53 were cloned into pLEX_305-C-dTAG and p53 as well as RelA were cloned into pLEX_305-N-dTAG using Gateway LR clonase II Enzyme mix kit (Invitrogen). The sequences of the primers are listed below.

Briefly, 100 ng pDONR221 vectors containing genes of interest were mixed with 150 ng of pLEX_305-C-dTAG or pLEX_305-N-dTAG in TE buffer. 1 ml of LR clonase II enzyme was added to the plasmid mix and incubated for 1 h at room temperature, followed by addition of 1 ml Proteinase K and incubated for 10 min at 37° C. to terminate the reaction. Samples were transformed using DH10B competent cells and plated on Ampicillin selective agar plates. After growing at 30° C. overnight, colonies were picked and grown up in 5 mL LB media supplemented with Ampicillin at 30° C. overnight. Plasmid DNA was purified using Zyppy Plasmid Miniprep Kit (Genesee Scientific) and sequenced using primers as followed:

(SEQ ID NO: 9)
pLEX_305-dTAG-seq-F: TGTTCCGCATTCTGCAAGCCTC
and
(SEQ ID NO: 10)
pLEX_305-dTAG-seq-R ACAAAGGCATTAAAGCAGCGTATCC

Lentivirus Production and Transduction: Lentiviral production was performed using HEK293T cells, which were co-transfected with pMD2.G (Addgene, #12259), psPAX2 (Addgene, #12260), and AceTAG lentiviral plasmids using PEI (Polysciences Inc). Viral particles were collected 72 h after transfection and filtered through a 0.45 mM membrane. A range of dilutions of the lentivirus in DMEM complete and 10 μg/mL polybrene were added to HeLa, H1299, or HeLa RelA KO cell lines. Transduced cells were selected with 1 μg/mL or 2 μg/mL puromycin.

Protein Expression: A construct containing residues 2-108 of FKBP12^{F36V} in pGEX4t-1 vector was overexpressed in E. coli BL21(DE3) in LB medium in the presence of 100 mg/mL carbenicillin. Cells were grown at 37° C. to an OD of 0.6, induced with 100 μM isopropyl-1-thio-D-galatopyranoside (IPTG), incubated for 4 h at 37° C., and collected by centrifugation. Cell pellets were suspended in lysis buffer (50 mM HEPES pH 8.0, 250 mM NaCl, 0.1%(v/v) Triton X-100, 1 mM TCEP), sonicated and then cleared by centrifugation at 11,000 g for 40 min at 4° C. FKBP12^{F36V} was first purified with Pro Affinity Concentration Kit GST (Amicon: ACR5000GS), and further purified on HiPrep 16/60 Sephacryl S-200 HR gel filtration column. Clean fraction was concentrated and buffer exchanged to storage buffer (50 mM HEPES, 250 mM NaCl, pH 8.0, 10% Glycerol), protein concentration was measured with Thermo 660 nM kit, flash freezed and stored at −80° C.

GST-FKBP12^{F36V}/His-P300-BRD AlphaScreen Assay: GST-FKBP12^{F36V} and 6xHis-p300-BRD (active motif #31372) were diluted to 250 nM and 500 nM, in assay buffer (50 mM HEPES pH 7.4, 200 mM NaCl, 1 mM TCEP, and 0.1% BSA), 20 mL of protein mixture was added to each well of a 1/2 area 96-well OptiPlate (PerkinElmer). Compounds were then added from DMSO stock to protein mixture (0.4 ml/well) with 1% DMSO in final mixture. Plates were then shaken orbitally at room temperature for 1 h. Nickel Chelate AlphaLISA Acceptor and Glutathione AlphaLISA Donor beads (PerkinElmer) diluted to 20 ng/ml in assay buffer and 20 ml beads mixture was added to each well. After a 1 h orbital shaking at room temperature. luminescence was measured on the Envision 2104 plate reader (PerkinElmer). Data were plotted and analyzed using GraphPad PRISM v8 and fit with ‘Bell-shaped’ dose response curve.

For ternary complex competition experiments, GST-FKBP12^{F36V} and 6xHis-p300-BRD (active motif #31372) were diluted to 250 nM and 500 nM, in assay buffer (50 mM HEPES pH 7.4, 200 mM NaCl, 1 mM TCEP, and 0.1% BSA). AceTAG-1 was added into protein mixture from 600 mM DMSO stock (final concentration 300 nM). 20 mL of protein mixture was added to each well of a 1/2 area 96-well OptiPlate (PerkinElmer). FKBP12^{F36V} competitor or p300-BRD competitor was then added from DMSO stock to protein mixture (0.4 ml/well) with 1% DMSO in final mixture. Plates were then shaken orbitally at room temperature for 1 h. Nickel Chelate AlphaLISA Acceptor and Glutathione AlphaLISA Donor beads (PerkinElmer) diluted to 20 ng/μl in assay buffer and 20 ml beads mixture was added to each well. After a 1 h orbital shaking at room temperature. luminescence was measured on the Envision 2104 plate reader (PerkinElmer) Data were plotted using GraphPad PRISM v8 and fit with ‘log(inhibitor)-response, four parameters’ curve.

AceTAG-1 Target Engagement: At 50%-70% confluency, HEK293t cells in 6-well plates were transfected with pLEX305-C-dTAG-H3.3. After 24 h, cells were co-treated for 30 min with 0.5 μM FKBP-p probe and increasing concentration of AceTAG-1 in serum-free DMEM media. Cells then underwent UV-photocrosslinking (365 nm) for 20 min at 4° C., harvested in cold DPBS by scraping and centrifugation, cell pellets were washed with cold DPBS two times and aspirated. Cells were then lysed in 400 μL DPBS buffer supplemented with 0.2% SDS (w/v) and 1X Halt protease inhibitor cocktail. Lysate was then normalized to 1.5 mg/mL. To each sample (50 μL), 6 μL of a freshly prepared “click” reagent mixture containing 0.1 mM tris(benzyltriazolylmethyl)amine (TBTA) (3 μL/sample, 1.7 mM in 1:4 DMSO:t-ButOH), 1 mM CuSO₄ (1 μL/sample, 50 mM in H₂O), 25 μM Rhodamine-azide (1 μL/sample, 1.25 mM in DMSO), and freshly prepared 1 mM tris(2-carboxyethyl)phosphine HCl (TCEP) (1 μL/sample, 50 mM in H₂O) was added to conjugate the fluorophore to probe-labeled proteins. Upon addition of the click mixture, each reaction was immediately mixed by vortexing and then allowed to react at room temperature for 1 h before quenching the reactions with SDS loading buffer (4X stock, 17 μL). Proteins (25 μg total protein loaded per gel lane) were resolved using SDS-PAGE (10% acrylamide) and visualized by in-gel fluorescence on Bio-Rad ChemiDoc MP Imaging System. Gel fluorescence and imaging was processed using Image Lab (v 6.0.1) software. Proteins were then transferred to PVDF membranes using Trans-Blot Turbo RTA Mini 0.45 mM LF PVDF Transfer Kit (Bio-Rad), and blotted with anti-HA antibody. Chemiluminescence was recorded with Bio-Rad ChemiDoc MP Imaging System and processed using Image Lab (v 6.0.1) software.

In-situ photocrosslinking with p300-BRD photocrosslinking probe and competition by AceTAG-1: At 50%-70% confluency, HEK293t cells in 6-well plates were transfected with pSG5-HA-p300. After 24 h, cells were co-treated for 30 min with 5 mM p300-p probe and increasing concentration of AceTAG-1 in serum-free media. Cells then underwent UV-photocrosslinking (365 nm) for 20 min at 4° C. and harvested in cold DPBS by scraping and centrifugation. Cell pellets were washed with cold DPBS two times and aspirated. Cells were then lysed in 200 μL DPBS buffer supplemented with 1X Halt protease inhibitor cocktail. Lysate was fractionated by ultracentrifuge (100000 g, 45 min, 4° C.) to provide soluble fraction (supernatant) and membrane fraction (pellets). Soluble fraction was normalized to 2 mg/mL, underwent click reaction with Rhodamine-azide as previously described. Proteins (25 μg total protein loaded per gel lane) were resolved using SDS-PAGE (6% acrylamide). In-gel fluorescence visualization and immunoblot analysis was carried out as previously described.

Immunoblotting: Cells were harvested in cold DPBS by scraping and washed with cold DPBS twice. Cell pellets were resuspended in DPBS supplemented with 5 mM sodium butyrate, 20 mM Nicotinamide, and 1X Halt Protease Inhibitor Cocktail and lysed by sonication (15 ms on, 40 ms off, 15% amplitude, 1s total on x 3). Protein concentration was determined using DC Protein Assay (Bio-Rad) and absorbance read using a CLARIOstar plate reader following manufacturer's instructions. Samples with equal protein content were boiled in 4X SDS gel loading buffer for 10 min. Proteins were separated by 10%, 12.5%, or 15% SDS-polyacrylamide gel electrophoresis and transferred to PVDF membranes using Trans-Blot Turbo RTA Mini 0.45 mM LF PVDF Transfer Kit (Bio-Rad). Membranes were incubated for 1 h at room temperature with blocking buffer, followed by incubating overnight at 4° C. with primary antibodies. After washing in TBST, the secondary antibodies were incubated with the membranes at room temperature for 1 h. The membranes were washed with TBST for three times and visualized on a Bio-Rad ChemiDoc MP Imaging System with Clarity Max Western ECL substrate (Bio-Rad) or SuperSignal West Femto Chemiluminescent substrate proprietary luminol and peroxide solution kit (Thermo Scientific Pierce).

Immunoprecipitation: Cells were split in 10 cm dishes in indicated media. The probes were added once the cells grew to 70-80% confluency and incubated for indicated time. The cells were harvested and washed with cold DPBS twice. The pelleted cells were lysed in 1 mL lysis buffer, containing 50 mM Tris-HCl pH 7.5, 300 mM NaCl, 0.5% (v/v) NP40, 5 mM sodium butyrate, 20 mM Nicotinamide, and 1X Halt Protease Inhibitor, followed by clarification by centrifugation at 16,000 g for 20 min at 4° C. The protein concentration was normalized by DC Proein Assay (Bio-Rad) and equal amount of protein was subjected to enrichment. For HA tag fusion protein enrichment, Monoclonal Anti-HA Agarose antibody produced in mouse (Sigma-Aldrich) was pre-washed with lysis buffer and added to the clarified lysate. Enrichment was carried out at 4° C. for 4 h with rotating. After allowing immune complex binding, the beads were spun down at 2,000 rpm for 3 min at 4° C. The beads were washed with lysis buffer and DPBS and incubated with HA peptide elution buffer at 37° C. for 15 min twice or 4X SDS gel loading buffer at 95° C. for 10 min. Supernatant was collected for immunoblot analysis or mass spectrometry sample preparation.

Chromatin Isolation: To isolate chromatin, 3×10⁶ cells were resuspended in 200 mL buffer A (10 mM HEPES, pH 7.9, 10 mM KCl, 1.5 mM MgCl₂, 0.34 M sucrose, 10% glycerol, 1 mM DTT, 5 mM sodium butyrate, 20 mM Nicotinamide, and 1X Halt Protease Inhibitor). Cells were incubated for 5 min on ice with 0.1% (v/v) Triton X-100. Nuclei and soluble protein were separated by low-speed centrifugation at 1,300 g for 15 min at 4° C. The soluble protein was further clarified by high-speed centrifugation at 20,000 g for 15 min at 4° C. Nuclei was washed with buffer A, following by lysis in buffer B (3 mM EDTA, 0.2 mM EGTA, 1 mM DTT, 5 mM sodium butyrate, 20 mM Nicotinamide, and 1X Halt Protease Inhibitor). Insoluble chromatin and nuclear plasma were separated by centrifugation at 1,700 g for 4 min at 4° C. The insoluble chromatin was washed once in buffer B and resuspended in DPBS with 0.1% SDS. Protein concentration of chromatin, nuclear plasma, and soluble protein were normalized by DC Protein Assay (Bio-Rad) and equal amount of protein was subjected to immunoblotting analysis as described above.

### Quantitative Proteomic Analysis of H3.3 Acetylation

Sample preparation: Cells were split in 10 cm dishes in indicated media. The probes were added once the cells grew to 60%-70% confluency and incubated for indicated time. The cells were harvested and washed with cold DPBS twice. The pelleted cells were lysed in 1 mL lysis buffer, containing 10 mM Tris pH 7.5, 300 mM NaCl, 0.1% (v/v) NP40, 5 mM sodium butyrate, 20 mM Nicotinamide, and 1X Halt Protease Inhibitor, followed by centrifuging for 5 min at 1,000 g at 4° C. The pellets were resuspended in benzonase buffer, containing 50 mM Tris pH 7.5, 300 mM NaCl, 0.5% NP40, and 2.5 mM MgCl₂. Protein concentration was normalized by DC Protein Assay (Bio-Rad) and equal amount of protein was subjected to 50 U/100 ml benzonase nuclease (Millipore Sigma) treatment.

Samples with benzonase were incubated on ice for 30 min and centrifuged at 300 g for 3 min at 4° C. to obtain soluble chromatin fraction. Samples were diluted with dilution buffer, containing 50 mM Tris pH 7.5, 300 mM NaCl, 0.5% NP40, and 15 mM EDTA to quench the benzonase activity. Protein concentration was normalized again by DC Protein Assay (Bio-Rad) and equal amount of protein was used for HA-tag protein enrichment as described above. The beads were washed with lysis buffer and DPBS and incubated with 8 M urea and boiled in 4X SDS gel loading buffer at 95° C. for 15 min. Supernatant was collected for in-gel digestion for mass-spectrometry sample preparation. HA-tag protein enriched samples were run on SDS-PAGE gel, flanked by MW markers, and stained with ProtoBlue. The desire protein bands were excised out and diced into approximately 1 mm³ pieces. The gels were washed with 500 ml 100 mM TEAB for 2 times and reduced in 10 mM TCEP solution at 60° C. for 30 min After reaction, TCEP solution was replaced with a solution of freshly prepared iodoacetamide (55 mM in 100 mM TEAB) and incubated for 30 min at room temperature while protected from light. A solution of 1:1 acetonitrile and 100 mM TEAB was added to wash the gels bands, followed by 100% acetonitrile to completely dry the gels. Sequencing-grade modified porcine trypsin (0.2 mg, 25 mM TEAB pH 8.5, 100 μM CaCl₂) was added to the gel and incubated at 37° C. for 14 h with shaking. The digest was collected and the peptides within the gels were extracted with 25% acetonitrile/5% formic acid (MS-grade) for one time, 75% acetonitrile (MS-grade) for two times, and 100% acetonitrile (MS-grade) for two times. The peptide samples were dried under vacuum centrifugation.

TMT Labeling and Fractionation: Samples were dissolved in 100 mM TEAB containing 30% acetonitrile (MS-grade) and labeled with respective TMT 10 plex isotope (8 mL, 20 μg/μL) for 1 h with occasional vortexing at RT. To quench the reaction, hydroxylamine (6 mL, 5% v v) was added to each sample, vortexed, and incubated for 15 min at RT. Formic acid (4 mL) was added to each tube to acidify and the samples were dried under vacuum centrifugation. Multiplexed samples were fractionated using the Pierce™ High pH Reversed-Phase Peptide Fractionation Kit according to manufacturer instructions with the following elution scheme (% acetonitrile in 0.1% TEA; F1: 5%, F2: 7.5%, F3: 10%, F4: 12.5%, F5: 15%, F6: 17.5%, F7: 20%, F8: 22.5%, F9: 25%, F10: 30%, F11: 50%, F12: 95%). Fractions were then combined into six fractions by the following scheme: F1+F7, F2+F8, F3+F9, F4+F10, F5+F11, F6+F12. Combined fractions were lyophilized prior to resuspension for MS analysis.

MS Analysis: TMT labeled samples were redissolved in MS buffer A (20 mL, 0.1% formic acid in water). 3 mL of each sample was loaded onto an Acclaim PepMap 100 precolumn (75 μm×2 mm) and eluted on an Acclaim PepMap RSLC analytical column (75 μm×15 cm) using the UltiMate 3000 RSLCnano system (Thermo Fisher Scientific). Buffer A (0.1% formic acid) and buffer B (0.1% formic acid in MeCN) were used in a 200 min gradient (flow rate 0.3 mL/min, 35° C.) of 2% buffer B for 10 min, followed by an incremental increase to 25% buffer B over 155 min, 25%-45% buffer B for 10 min, 45%-95% buffer B for 5 min, hold at 95% buffer B for 2 min, followed by descent to 2% buffer B for 1 min and re-equilibration at 2% for 6 min. The elutions were analyzed with a Thermo Fisher Scientific Orbitrap Fusion Lumos mass spectrometer with a cycle time of 3 s and nano-LC electrospray ionization source applied voltage of 2.0 kV. MS¹ spectra were recorded at a resolution of 120,000 with an automatic gain control (AGC) value of 1×10⁶ ions, maximum injection time of 50 ms (dynamic exclusion enabled, repeat count 1, duration 20 s). The scan range was specified from 375 to 1,500 m/z. Peptide fragmentation MS² spectra was recorded via collision-induced diffusion (CID) and quadrupole ion trap analysis (AGC 1.8×10⁴, 30% collision energy, maximum inject time 120 ms, isolation window 0.7). MS³ spectra were generated by high-energy collision-induced dissociation (HCD) with collision energy of 65%. Precursor selection included up to 10 MS²ions for the MS³ spectrum. Proteomic analysis was performed with the processing software Proteome Discoverer 2.4 (Thermo Fisher Scientific). Peptide sequences were identified by matching proteome databases with experimental fragmentation patterns via the SEQUEST HT algorithm. Fragment tolerances were set to 0.6 Da, and precursor mass tolerances set to 10 ppm with four missed cleavage sites allowed. Carbamidomethyl (C, +57.021) and TMT-tag (N-terminal, +229.163) were specified in the static modifications. Oxidation (M, +15.995), TMT-tag (K, +229.163), and Acetylation (K, +42.011) were defined as dynamic modifications. Spectra were searched against the Homo sapiens proteome database (42,358 sequences) using a false discovery rate of 1% (Percolator). MS³ peptide quantitation was performed with a mass tolerance of 20 ppm. Peptide abundance of TMT ratios obtained by Proteome Discoverer were normalized to total protein abundance.

### Quantitative Acetylproteomics

Sample preparation: Frozen cell pellets were thawed on ice and lysed in a buffer containing 50 mM Tris-HCl (pH 7.5), 150 mM NaCl, 1 mM EDTA, 1% NP-40, 0.1% sodium deoxycholate, 5 mM sodium butyrate, 10 mM nicotinamide and 1X Pierce™ protease inhibitor cocktail. Cells were sonicated to ensure complete lysis, mixed with 5 M NaCl at a ratio of 1:10 with sample volume and incubated on ice for 15 min to release chromatin-bound proteins. Cell lysates were then sonicated again to shear genomic DNA. Cellular debris was cleared by centrifugation at 16,000 g for 20 min at 4° C., protein concentration was determined using a Pierce™ BCA assay and ˜10 mg of protein was aliquoted for acetyl-proteomic analysis. Proteins were denatured with 8 M urea in 50 mM HEPES, reduced with 5 mM dithiothreitol for 30 min at 56° C., then alkylated with 5 mM iodoacetamide for 20 min in the dark at RT. Following alkylation, proteins were precipitated using chloroform-methanol as previously described, and protein pellets were dried at 56° C. for 15 min. Proteins pellets were resuspended in 1 M urea in 50 mM HEPES and digested in a two-step process with LysC and trypsin as previously described. Digested peptides were desalted using Phenomenex Strata-X polymeric reverse phase extraction cartridges using the following protocol. Cartridges were conditioned with 1 mL 100% acetonitrile, 1 mL 80% acetonitrile/0.5% AA, and 1 mL 0.1% TFA. Peptides were then loaded on to the cartridge at a reduced flow rate and desalted with 5 mL 0.1% TFA followed by 1 mL 0.5% AA. Desalted peptides were eluted using 1 mL 40% acetonitrile/0.5% AA followed by 1 mL 80% acetonitrile/0.5% AA and lyophilized. Digested peptides were quantified using the Pierce™ Quantitative Colorimetric Peptide Assay and 25 mg was aliquoted from each for total protein analysis while the remaining peptide was lyophilized and retained for acetyl-peptide enrichment.

Acetyl-peptide Enrichment: Acetyl-peptides were enriched using the PTMScan® Acetyl-Lysine Motif [Ac-K] Kit according to manufacturer instructions. Briefly, lyophilized peptides were resuspended in immuno-affinity purification buffer (IAP) and added to PBS-washed antibody-bead slurry. Acetyl-peptides were enriched on a rotator for 2 h at 4° C. Bead-peptide complexes were then washed 2X with 1 mL IAP and 3X with 1 mL H2O. Acetyl-peptides were eluted from the beads with 2X treatments of 0.15% TFA (gently mixing at RT for 10 min for each elution), desalted with Phenomenex Strata-X polymeric reverse phase extraction cartridge as above and lyophilized prior to TMT labeling.

TMT Labeling and Fractionation: Peptide aliquots were resuspended in 30% dry acetonitrile, 200 mM HEPES, pH 8.5 and 8 ml of TMT labeling reagents was added to each sample. Labeling was allowed to proceed for 1 h at RT after which TMT labels were quenched by the addition of 9 μl 5% hydroxylamine for 15 min before the samples were acidified with 50 μl 1% TFA. Samples were then pooled into respective 10plexes, lyophilized and resuspended in 0.1% TFA. Multiplexed samples were fractionated using the Pierce™ High pH Reversed-Phase Peptide Fractionation Kit according to manufacturer instructions with the following elution scheme (% acetonitrile in 0.1% TEA; F1: 7.5%, F2: 10%, F3: 12.5%, F4: 15%, F5: 17.5%, F6: 20%, F7: 22.5%, F8: 25%, F9: 27.5%, F10: 30%, F11: 32.5%, F12: 35%, F13: 37.5%, F14: 40%, F15: 42.5%, F16: 45%, F17: 47.5%, F18: 75%). Fractions were then combined into nine fractions by the following scheme: F1+F10, F2+F11, F3+F12, F4+F13, F5+F14, F6+F15, F7+F16, F8+F17, F9+18. Combined fractions were lyophilized prior to resuspension for MS analysis.

MS analysis: Acetylproteomic samples were subject to analysis via LC-MS³ as above with the following alterations. Samples were resuspended in 60 mL buffer A and 10 mL was loaded on to the analytical column. Samples were eluted with 5% buffer B for 10 min, a gradient of 5-20% buffer B over 160 min, 20-45% buffer B over 20 min, 45-95% buffer B over 5 min, 95% buffer B for 2 min, 5% buffer B for 2 min, 95% buffer B for 2 min and 5% buffer B for 10 min to re-equilibrate the column. Eluted peptides were analyzed with a Thermo Fisher Orbitrap Fusion mass spectrometer as above with the following alterations: MS¹ scan range of 400-1700 m/z, dynamic exclusion of 15 sec, MS1 AGC target of 2×10⁵, MS² CID collision energy 35%, MS³ HCD collision energy of 55%.

Chemistry Materials: Chemicals and reagents were purchased from commercial vendors, including Sigma-Aldrich, Fisher Scientific, Combi-Blocks, MedChemExpress, Alfa Aesar and AstaTech, and were used as received without further purification, unless otherwise noted. Anhydrous solvents were purchased from Sigma-Aldrich in Sure/Seal™ formulations. All reactions were monitored by thin-layer chromatography (TLC, Merck silica gel 60 F-254 plates). The plates were stained either with p-anisaldehyde (2.5% p-anisaldehyde, 1% AcOH, 3.5% H2SO4 (conc.) in 95% EtOH), ninhydrin (0.3% ninhydrin (w/v), 97:3 EtOH-AcOH), KMnO4 (1.5 g of KMnO4, 10 g K2CO3, and 1.25 mL 10% NaOH in 200 mL water), iodine or directly visualized with UV light. Reaction purification was carried out using Flash chromatography (230-400 mesh silica gel), Biotage® or thin layer chromatography (pTLC, Analtech, 500-2000 μm thickness). NMR spectra were recorded on Bruker DPX-400 MHz or Bruker AV-600 MHz spectrometers in the indicated solvent. Multiplicities are reported with the following abbreviations: s singlet; d doublet; t triplet; q quartet; p pentet; m multiplet; br broad; dd doublet of doublets; dt doublet of triplets; td triplet of doublets; Chemical shifts are reported in ppm relative to the residual solvent peak and J values are reported in Hz. Mass spectrometry data were collected on an Agilent 6120 single-quadrupole LC/MS instrument (ESI, low resolution) or an Agilent ESI-TOF instrument (ESI-TOF, HRMS).

### Compound Synthesis and Characterization:

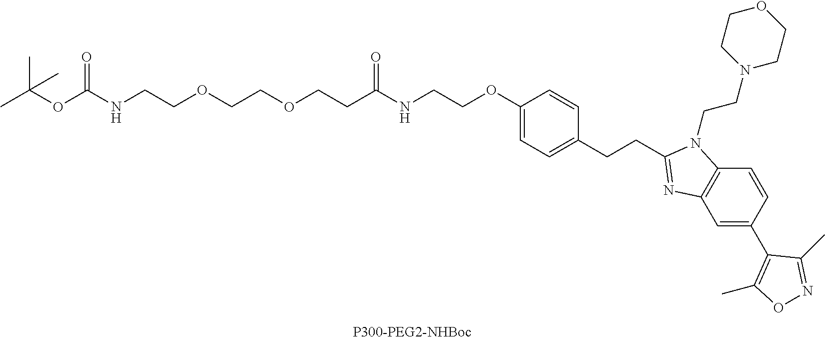 [SMILES: Cc1noc(C)c1-c1ccc2c(c1)nc(CCc1ccc(OCCNC(=O)CCOCCOCCNC(=O)OC(C)(C)C)cc1)n2CCN1CCOCC1]

tert-butyl (2-(2-(3-((2-(4-(2-(5-(3,5-dimethylisoxazol-4-yl)-1-(2-morpholinoethyl)-1H-benzo[d]imidazol-2-yl)ethyl)phenoxy)ethyl)amino)-3- oxopropoxy)ethoxy)ethyl)carbamate (P300-PEG2-NHBoc): To a solution of 2-(4-(2-(5-(3,5-dimethylisoxazol-4-yl)-1-(2-morpholinoethyl)-1H-benzo[d]imidazol-2-yl)ethyl)phenoxy)ethan-1-amine (10.0 mg, 0.020 mmol, 1.0 eq) (synthesized according to previous method¹) in DMF (1 mL), DIPEA (10.6 μL, 0.061 mmol, 3.0 eq) and t-Boc-N-amido-PEG2-acid (5.35 mg, 0.020 mmol, 1 eq) was added followed by HATU (8.54 mg, 0.022 mmol, 1.1 eq) were added at 0° C. and resulting mixture was stirred for 5 minutes the corresponding starting material was fully consumed (indicated by TLC). The crude mixture was diluted with cold water and extracted in ethyl acetate (20 mL×3) then combined organic extract was dried over sodium sulfate, filtered, and concentrated under reduced pressure. The crude material was purified by PTLC (DCM/Methanol, 19:1) to obtain P300-PEG2-NHBoc as a colorless sticky material (8.4 mg, 55%).

¹H NMR: (400 MHz, CDCl₃) δ 7.62 (dd, J=1.5, 0.6 Hz, 1H), 7.35 (dd, J=8.3, 0.7 Hz, 1H), 7.18-7.10 (m, 3H), 6.88-6.80 (m, 3H), 4.13 (t, J=6.9 Hz, 2H), 4.02 (t, J=5.3 Hz, 2H), 3.75-3.63 (m, 10H), 3.62-3.56 (m, 4H), 3.27-3.20 (m, 2H), 3.19-3.13 (m, 2H), 2.62 (t, J=6.8 Hz, 2H), 2.52-2.44 (m, 8H), 2.42 (s, 3H), 2.29 (s, 3H), 1.43 (s, 9H).

¹³C NMR: (101 MHz, CDCl₃) δ 171.87, 170.88, 165.04, 159.02, 157.32, 155.38, 143.00, 134.27, 133.34, 129.41, 124.27, 123.46, 119.92, 117.10, 114.71, 109.44, 80.67, 70.24, 70.11, 67.19, 66.89, 66.84, 57.64, 54.06, 41.52, 38.88, 36.94, 36.22, 32.98, 29.88, 28.11, 11.60, 10.90.

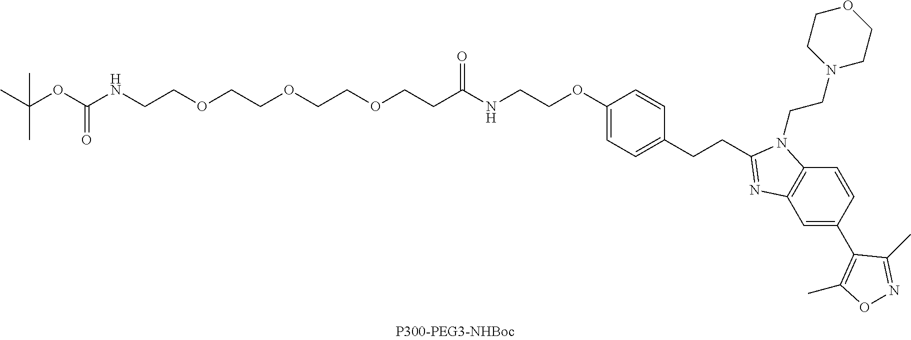 [SMILES: Cc1noc(C)c1-c1ccc2c(c1)nc(CCc1ccc(OCCNC(=O)CCOCCOCCOCCNC(=O)OC(C)(C)C)cc1)n2CCN1CCOCC1]

tert-butyl (15-(4-(2-(5-(3,5-dimethylisoxazol-4-yl)-1-(2-morpholinoethyl)-1H-benzo[d]imidazol-2-yl)ethyl)phenoxy)-12-oxo-3,6,9-trioxa-13- azapentadecyl)carbamate (P300-PEG3-NHBoc)

To a solution of 2-(4-(2-(5-(3,5-dimethylisoxazol-4-yl)-1-(2-morpholinoethyl)-1H-benzo[d]imidazol-2-yl)ethyl)phenoxy)ethan-1-amine (10.0 mg, 0.020 mmol, 1.0 eq) (synthesized according to previous method¹) in DMF (1 mL), DIPEA (10.6 μL, 0.061 mmol, 3.0 eq) and t-Boc-N-amido-PEG3-acid (6.56 mg, 0.020 mmol, 1 eq) was added followed by HATU (8.54 mg, 0.022 mmol, 1.1 eq) were added at 0° C. and resulting mixture was stirred for 5 minutes the corresponding starting material was fully consumed (indicated by TLC). The reaction mixture was diluted with cold water and extracted in ethyl acetate (20 mL×3) then combined organic extract was dried over sodium sulfate, filtered, and concentrated under reduced pressure. The crude material was purified by PTLC (DCM/Methanol, 19:1) to obtain P300-PEG2-NHBoc as a colorless sticky material (8.5 mg, 52%).

¹H NMR: (600 MHz, CDCl₃) δ 7.62 (d, J=1.5 Hz, 1H), 7.35 (d, J=8.3 Hz, 1H), 7.16-7.13 (m, 2H), 7.12 (dd, J=8.2, 1.5 Hz, 1H), 6.84 (d, J=8.6 Hz, 2H), 6.76 (s, 1H), 5.07 (s, 1H), 4.13 (t, J=6.9 Hz, 2H), 4.02 (t, J=5.3 Hz, 2H), 3.74 (t, J=5.8 Hz, 2H), 3.68-3.61 (m, 10H), 3.60 (s, 4H), 3.52 (t, J=5.1 Hz, 2H), 3.33-3.27 (m, 2H), 3.22-3.13 (ddd, J=9.7, 5.1, 2.1 Hz, 2H), 3.17 (ddd, J=8.5, 7.0, 2.2 Hz, 2H), 2.62 (t, J=6.9 Hz, 2H), 2.51 (t, J=5.8 Hz, 2H), 2.46 (t, J=4.6 Hz, 4H), 2.42 (s, 3H), 2.29 (s, 3H), 1.42 (s, 9H).

¹³C NMR: (151 MHz, CDCl₃) δ 171.92, 165.06, 159.03, 157.26, 155.39, 142.97, 134.27, 133.36, 129.42, 124.25, 123.47, 119.89, 117.11, 114.71, 109.47, 70.56, 70.35, 70.20, 70.16, 70.04, 67.24, 66.84, 66.76, 57.63, 54.05, 41.51, 40.34, 38.92, 32.97, 29.86, 28.42, 11.61, 10.92.

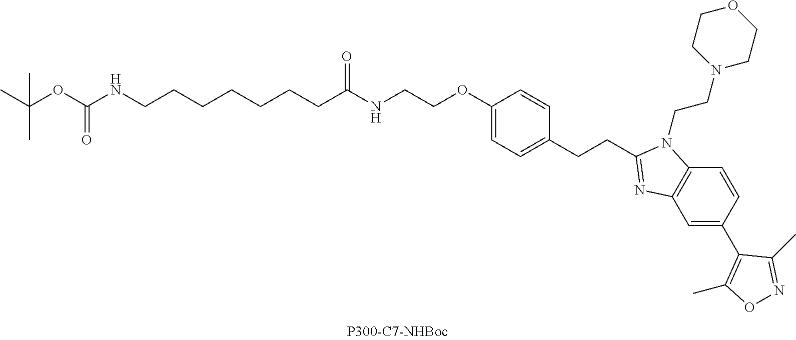 [SMILES: Cc1noc(C)c1-c1ccc2c(c1)nc(CCc1ccc(OCCNC(=O)CCCCCCCNC(=O)OC(C)(C)C)cc1)n2CCN1CCOCC1]

tert-butyl (8-((2-(4-(2-(5-(3,5-dimethylisoxazol-4-yl)-1-(2-morpholinoethyl)-1H-benzo[d]imidazol-2-yl)ethyl)phenoxy)ethyl)amino)-8-oxooctyl)carbamate (P300-C7-NHBoc)

To a solution of 2-(4-(2-(5-(3,5-dimethylisoxazol-4-yl)-1-(2-morpholinoethyl)-1H-benzo[d]imidazol-2-yl)ethyl)phenoxy)ethan-1-amine (30.0 mg, 0.061 mmol, 1.0 eq) (synthesized according to previous method¹) and Boc-8-Aoc-OH (17.5 mg, 0.067 mmol, 1.1 eq) in DCM (4 mL), EDC (18.0 mg, 0.092 mmol, 1.5 eq) HOBt (13.0 mg, 0.092 mmol, 1.5 eq) and DIPEA (32 μL, 0.183 mmol, 3.0 eq) was added. The mixture was stirred at room temperature for 14 hours. After completion the reaction mixture was diluted with water (10 mL) and extracted in DCM (2×20 mL), the organic layer was washed with saturated sodium bicarbonate (aq), and brine, the combined organic layer was dried over sodium sulfate, filtered, and concentrated under reduced pressure. The crude material was purified by Biotage® Sfär Silica D 10 g column with a 0-5% linear gradient of Methanol in Dichloromethane over 20 column volumes (CV), to obtain P300-C7-NHBoc as a colorless oil (29 mg, 65%).

¹H NMR (600 MHZ, CDCl₃) δ 7.62 (d, J=1.5 Hz, 1H), 7.36 (s, 1H), 7.18-7.15 (m, 2H), 7.12 (dd, J=8.2, 1.5 Hz, 1H), 6.86-6.82 (m, 2H), 5.93 (t, J=5.9 Hz, 1H), 4.51 (s, 1H), 4.13 (t, J=6.9 Hz, 2H), 4.01 (t, J=5.1 Hz, 2H), 3.66 (td, J=4.9, 2.8 Hz, 6H), 3.27-3.21 (m, 2H), 3.20-3.14 (m, 2H), 3.08 (q, J=6.7 Hz, 2H), 2.62 (t, J=6.9 Hz, 2H), 2.46 (t, J =4.6 Hz, 4H), 2.42 (s, 3H), 2.29 (s, 3H), 2.21-2.16 (m, 2H), 1.63 (t, J=7.3 Hz, 3H), 1.43 (s, 9H), 1.33-1.28 (m, 6H).

¹³C NMR (151 MHz, CDCl₃) δ 173.26, 165.06, 159.03, 157.18, 156.01, 155.35, 143.03, 134.28, 133.51, 129.47, 124.26, 123.46, 119.95, 117.10, 114.66, 109.42, 66.94, 66.84, 57.64, 54.06, 41.50, 40.52, 38.89, 36.65, 32.94, 29.98, 29.87, 29.72, 29.11, 28.91, 28.44, 26.56, 25.52, 11.61, 10.92.

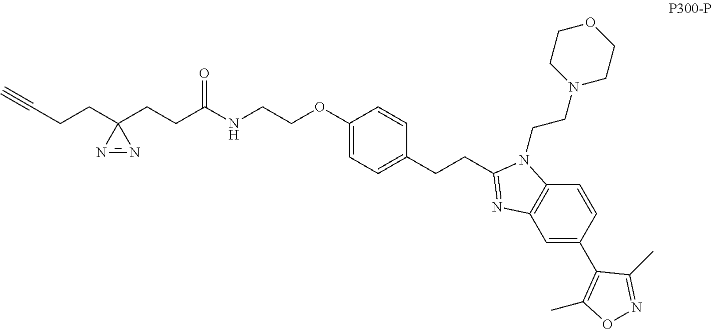 [SMILES: C#CCCC1(CCC(=O)NCCOc2ccc(CCc3nc4cc(-c5c(C)noc5C)ccc4n3CCN3CCOCC3)cc2)N=N1]

3-(3-(but-3-yn-1-yl)-3H-diazirin-3-yl)-N-(2-(4-(2-(5-(3,5-dimethylisoxazol-4-yl)-1-(2-morpholinoethyl)-1H-benzo[d]imidazol-2- yl)ethyl)phenoxy)ethyl)propenamide (P300-P)

To a solution of 2-(4-(2-(5-(3,5-dimethylisoxazol-4-yl)-1-(2-morpholinoethyl)-1H-benzo[d]imidazol-2-yl)ethyl)phenoxy)ethan-1-amine (15.0 mg, 0.030 mmol, 1.0 eq) (synthesized according to previous method¹) and 3-(3-(but-3-yn-1-yl)-3H-diazirin-3-yl)propanoic acid (6.0 mg, 0.033 mmol, 1.1 eq) in DCM (2 mL), EDC (9.0 mg, 0.046 mmol, 1.5 eq) HOBt (7.0 mg, 0.046 mmol, 1.5 eq) and DIPEA (16 μL, 0.092 mmol, 3.0 eq) was added. The mixture was stirred at room temperature for 12 hours, then diluted with DCM. The organic layer was washed with saturated sodium bicarbonate (aq), water and brine. The combined organic layer was then dried over sodium sulfate, filtered, and concentrated under reduced pressure. The crude material was purified by Biotage® Sfär Silica D 10 g with a 0-5% linear gradient of Methanol in Dichloromethane over 20 column volumes (CV), to obtain P300-diazirine tag as a colorless oil (12.7 mg, 65%).

¹H NMR (400 MHz, CDCl₃) δ 7.62 (d, J=1.6 Hz, 1H), 7.35 (d, J=8.3 Hz, 1H), 7.19-7.09 (m, 3H), 6.86-6.79 (m, 2H), 6.10-5.91 (m, 1H), 4.13 (t, J=6.8 Hz, 2H), 4.01 (t, J=5.1 Hz, 2H), 3.70-3.61 (m, 6H), 3.23 (dd, J=8.9, 5.8 Hz, 2H), 3.20-3.12 (m, 2H), 2.61 (t, J=6.8 Hz, 2H), 2.45 (t, J=4.6 Hz, 4H), 2.42 (s, 3H), 2.29 (s, 3H), 2.03-1.93 (m, 5H), 1.84 (dd, J=8.9, 6.6 Hz, 2H), 1.63 (t, J=7.3 Hz, 2H).

¹³C NMR (101 MHz, CDCl₃) δ 171.30, 165.04, 159.02, 157.09, 155.34, 143.03, 134.28, 133.56, 129.47, 124.22, 123.45, 119.92, 117.10, 114.63, 109.43, 82.71, 69.26, 66.83, 66.73, 57.64, 54.05, 41.50, 39.04, 32.92, 32.35, 30.31, 29.85, 28.35, 27.85, 13.28, 11.60, 10.92.

HRMS (ESI-TOF) calcd for C₃₆H₄₄N₇O₄, 638.3449 (M+H⁺), found 638.3451.

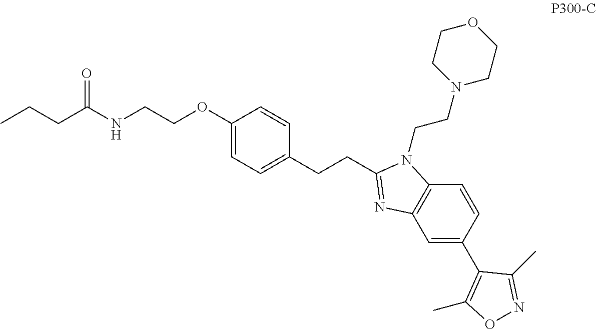 [SMILES: CCCC(=O)NCCOc1ccc(CCc2nc3cc(-c4c(C)noc4C)ccc3n2CCN2CCOCC2)cc1]

N-(2-(4-(2-(5-(3,5-dimethylisoxazol-4-yl)-1-(2-morpholinoethyl)-1H-benzo[d]imidazol-2-yl)ethyl)phenoxy)ethyl)pentanamide (P300-C)

To a solution of 2-(4-(2-(5-(3,5-dimethylisoxazol-4-yl)-1-(2-morpholinoethyl)-1H-benzo[d]imidazol-2-yl)ethyl)phenoxy)ethan-1-amine (synthesized according to previous method¹) (15.0 mg, 0.030 mmol, 1.0 eq) and butyric acid (3.0 mg, 0.034 mmol, 1.1 eq) in DCM (2 mL), EDC (9.0 mg, 0.046 mmol, 1.5 eq) HOBt (7.0 mg, 0.046 mmol, 1.5 eq) and DIPEA (16 μL, 0.092 mmol, 3.0 eq) was added. The mixture was stirred at room temperature for 12 hours, then diluted with DCM. The organic layer was washed with saturated sodium bicarbonate (aq), water and brine. The combined organic layer was then dried over sodium sulfate, filtered, and concentrated under reduced pressure. The crude material was purified by PTLC (DCM/Methanol, 19:1) to afford P300-C as a colorless oil (10.0 mg, 58%).

¹H NMR: (400 MHz, MeOD) δ 7.61-7.54 (m, 2H), 7.24 (dd, J=8.3, 1.6 Hz, 1H), 7.17-7.09 (m, 2H), 6.86 (d, J=8.6 Hz, 2H), 4.20 (t, J=6.7 Hz, 2H), 4.01 (t, J=5.5 Hz, 2H), 3.64 (t, J=4.7 Hz, 4H), 3.55 (t, J=5.5 Hz, 2H), 3.27- 3.24 (m, 2H), 3.23-3.14 (m, 2H), 2.54 (t, J=6.7 Hz, 2H), 2.49-2.41 (m, 7H), 2.29 (s, 3H), 2.20 (dd, J=7.9, 6.9 Hz, 2H), 1.69-1.60 (m, 2H), 0.94 (t, J=7.4 Hz, 3H).

¹³C NMR: (151 MHz, MeOD) δ 175.06, 165.34, 158.83, 157.56, 156.11, 142.02, 134.11, 132.96, 129.19, 124.07, 123.51, 118.43, 116.96, 114.36, 110.25, 66.45, 66.15, 57.06, 53.63, 40.86, 38.67, 37.49, 32.95, 29.16, 18.96, 12.53, 10.00, 9.33.

HRMS (ESI-TOF) calculated for C₃₂H₄₁N₅O₄, 560.3231 (M+H⁺), found 560.3247.

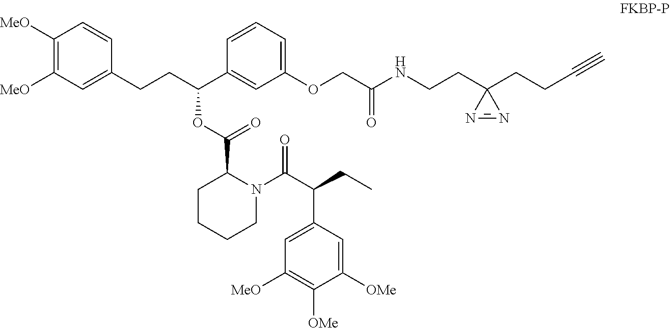 [SMILES: C#CCCC1(CCNC(=O)COc2cccc([C@@H](CCc3ccc(OC)c(OC)c3)OC(=O)[C@@H]3CCCCN3C(=O)[C@@H](CC)c3cc(OC)c(OC)c(OC)c3)c2)N=N1]

3) (R)-1-(3-(2-((2-(3-(but-3-yn-1-yl)-3H-diazirin-3-yl)ethyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (FKBP-P)

To a solution of 2-(3-((R)-3-(3,4-dimethoxyphenyl)-1-(((S)-1-((S)-2-(3,4,5-trimethoxyphenyl)butanoyl)piperidine-2-carbonyl)oxy)propyl)phenoxy)acetic acid (synthesized according to previous method²) (10.0 mg, 0.014 mmol, 1.0 eq) and 2-(3-(but-3-yn-1-yl)-3H-diazirin-3-yl)ethan-1-amine (2.20 mg, 0.016 mmol, 1.1 eq) in DCM (2 mL), EDC (4.15 mg, 0.022 mmol, 1.5 eq), HOBt (2.94 mg, 0.022 mmol, 1.5 eq) and DIPEA (8.0 μL, 0.043 mmol, 3.0 eq) was added. The mixture was stirred at room temperature for 12 hours, after completion the reaction mixture was diluted with DCM (15 mL). The organic layer was washed with saturated sodium bicarbonate (aq), water and brine, the combined organic layer was dried over sodium sulfate, filtered, and concentrated under reduced pressure. The crude material was purified by PTLC (DCM/Methanol, 19:1) to afford FKBP-P as a colorless oil (8.0 mg, 58%).

¹H NMR (600 MHz, CDCl₃) δ 7.33 (t, J=7.9 Hz, 0.25H), 7.19 (t, J=7.9 Hz, 1H), 7.01-6.99 (m, 0.25H), .6.93- 6.92 (m, 0.25H), 6.88-6.86 (m, 0.46H), 6.82-6.80 (m, 1.60H), 6.79-6.76 (m, 0.25H), 6.69-6.66 (m, 1.37H), 6.66-6.62 (m, 1.20H), 6.42 (s, 0.5H), 6.40 (s, 2H), 5.81 (dd, J=7.8, 6.0 Hz, 0.23H), 5.63 (dd, J=8.3, 5.4 Hz, 1H), 5.47-5.44 (m, 1H), 4.66 (d, J=5.4 Hz, 0.22H), 4.61-4.58 (m, 0.26H), 4.50 (s, 0.5H), 4.48 (s, 2H), 3.87-3.83 (m, 10H), 3.82-3.78 (m, 1H), 3.78 (s, 3H), 3.67 (s, 6H), 3.59-3.57 (m, 1H), 3.39-3.37 (m, 0.26H), 3.28-3.21 (m, 2.5H), 2.79 (td, J=13.4, 3.1 Hz, 1H), 2.63-2.53 (m, 1.5H), 2.50-2.44 (m, 1H), 2.42-2.40 (m, 0.23H), 2.30 (dd, J=12.0, 2.6 Hz, 1H), 2.13-2.04 (m, 2.61H), 2.02-1.98 (m, 4H), 1.96-1.91 (m, 1H), 2.04 1.77-1.67 (m, 6H), 1.66-1.61 (m, 3H), 1.47-1.39 (m, 1H), 1.31-1.25 (m, 3H), 0.90 (t, J=7.3 Hz, 3H), 0.86 (t, J=7.3 Hz, 1H). Note: rotomeric isomers observed.

¹³C NMR (101 MHz, CDCl₃) δ 172.67, 170.65, 168.26, 157.31, 153.51, 153.20, 148.89, 147.37, 142.41, 136.64, 135.32, 133.34, 130.14, 129.85, 120.21, 120.11, 120.07, 119.92, 113.79, 113.72, 113.55, 113.08, 111.69, 111.59, 111.34, 111.27, 104.98, 104.58, 82.60, 75.60, 69.45, 67.37, 67.31, 60.92, 60.80, 56.33, 55.97, 55.94, 55.88, 55.86, 55.69, 52.06, 51.25, 50.81, 43.49, 39.69, 38.24, 38.01, 34.05, 34.02, 32.66, 32.63, 32.05, 31.46, 31.30, 29.72, 28.40, 28.37, 26.79, 26.74, 26.36, 25.34, 24.54, 20.91, 20.68, 13.22, 12.79, 12.58. Note: rotomeric isomers observed.

HRMS (ESI-TOF) calculated for C₄₅H₅₇N₄O₁₀, 813.4069 (M+H⁺), found 813.4072.

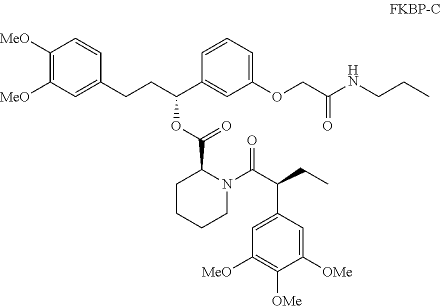 [SMILES: CCCNC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

(R)-3-(3,4-dimethoxyphenyl)-1-(3-(2-oxo-2-(propylamino)ethoxy)phenyl)propyl (S)-1-((S)-2-(3,4,5-trimethoxyphenyl)butanoyl)piperidine-2-carboxylate (FKBP-C)

To a solution of 2-(3-((R)-3-(3,4-dimethoxyphenyl)-1-(((S)-1-((S)-2-(3,4,5-trimethoxyphenyl)butanoyl)piperidine-2-carbonyl)oxy)propyl)phenoxy)acetic acid (synthesized according to previous method²) (15.0 mg, 0.030 mmol, 1.0 eq) and Propylamine (1.3 μL, 0.034 mmol, 1.1 eq) in DCM (2 mL), EDC (9.0 mg, 0.046 mmol, 1.5 eq), HOBt (7.0 mg, 0.046 mmol, 1.5 eq) and DIPEA (16 μL, 0.092 mmol, 3.0 eq) was added. The mixture was stirred at room temperature for 12 hours. After completion the reaction mixture was diluted with DCM (15 mL), the organic layer was washed with saturated sodium bicarbonate (aq), water and brine. The combined organic layer was dried over sodium sulfate, filtered, and concentrated under reduced pressure. The crude material was purified by PTLC (DCM/Methanol, 19:1) to obtain P300-C as a colorless oil (5.8 mg, 54%).

¹H NMR (400 MHz, MeOD) δ 7.34 (t, J=7.8 Hz, 0.21H), 7.20 (t, J=7.9 Hz, 1H), 7.07-6.94 (m, 1H), 6.95-6.84 (m, 2.24H), 6.81-6.79 (m, 1.28H), 6.77-6.72 (m, 1.36H), 6.69 (dd, J=8.2, 2.0 Hz, 1H), 6.63-6.55 (m, 3.30H), 5.84 (dd, J=7.9, 5.7 Hz, 0.2H), 5.61 (dd, J=8.3, 5.4 Hz, 1H), 5.45-5.35 (m, 1H), 4.98-4.90 (m, 1H), 4.54 (s, 0.5H), 4.51 (s, 2H), 4.14-4.05 (m, 1.25H), 3.93-3.85 (m, 2H), 3.84 (s, 1.38H), 3.82 (s, 3H), 3.81 (s, 3H), 3.77 (s, 1H), 3.71 (s, 3H), 3.70-3.65 (m, 6H), 3.61-3.58 (m, 0.5H), 3.30-3.22 (m, 2.5H), 2.78-2.70 (m, 1.22H), 2.67-2.53 (m, 2H), 2.49-2.41 (m, 1.5H), 2.33-2.29 (m, 1.3H), 2.20-2.11 (m, 0.6H), 2.10-1.98 (m, 3H), 1.97-1.85 (m, 1.5H), 1.79-1.85 (m, 5H), 1.56-1.48 (m, 4H), 1.37-1.24 (m, 4H), 0.94-0.89 (m, 6H), 0.85 (t, J=7.3 Hz, 1H). Note: rotomeric isomers observed.

¹³C NMR (101 MHz, MeOD) δ 173.76, 173.60, 170.48, 170.02, 169.51, 158.00, 157.79, 153.48, 153.19, 149.05, 147.48, 142.21, 136.55, 136.13, 135.52, 133.81, 133.75, 129.60, 129.44, 120.39, 120.31, 119.69, 119.30, 114.13, 113.68, 113.16, 112.89, 112.27, 111.88, 105.19, 104.61, 76.72, 75.74, 66.89, 59.68, 55.96, 55.15, 55.06, 52.23, 50.24, 49.87, 43.57, 37.84, 31.20, 30.86, 28.00, 26.21, 24.91, 20.44, 19.63, 12.70, 11.58, 11.27. Note: rotomeric isomers observed.

HRMS calculated for C₄₁H₅₅N₂O₁₀, 749.4008 (M+H⁺), found 749.4010.

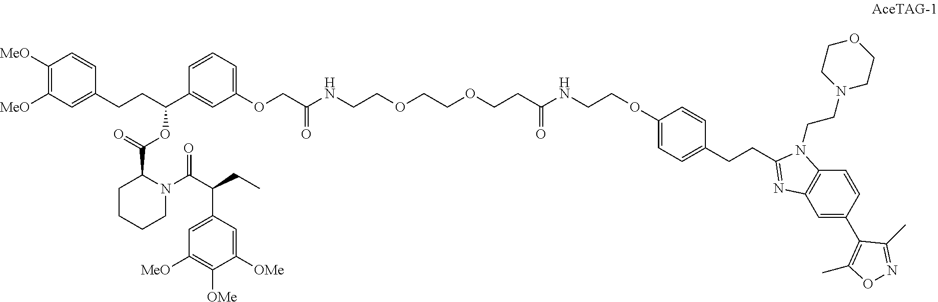 [SMILES: CC[C@H](C(=O)N1CCCC[C@H]1C(=O)O[C@H](CCc1ccc(OC)c(OC)c1)c1cccc(OCC(=O)NCCOCCOCCC(=O)NCCOc2ccc(CCc3nc4cc(-c5c(C)noc5C)ccc4n3CCN3CCOCC3)cc2)c1)c1cc(OC)c(OC)c(OC)c1]

(R)-3-(3,4-dimethoxyphenyl)-1-(3-((15-(4-(2-(5-(3,5-dimethylisoxazol-4-yl)-1-(2-morpholinoethyl)-1H-benzo[d]imidazol-2-yl)ethyl)phenoxy)- 2,12-dioxo-6,9-dioxa-3,13-diazapentadecyl)oxy)phenyl)propyl (S)-1-((S)-2-(3,4,5-trimethoxyphenyl)butanoyl)piperidine-2-carboxylate (AceTAG-1):

GTo a solution of tert-butyl (2-(2-(3-((2-(4-(2-(5-(3,5-dimethylisoxazol-4-yl)-1-(2-morpholinoethyl)-1H-benzo[d]imidazol-2-yl)ethyl)phenoxy)ethyl)amino)-3- oxopropoxy)ethoxy)ethyl)carbamate (P300-PEG2-NHBoc) (20 mg, 0.027 mmol) in DCM (1 mL), a solution of 20% TFA in DCM (1 mL) was added at 0° C. and resulting mixture was stirred at room temperature for 2 h. After completion (monitored by TLC), the reaction mixture was evaporated under reduced pressure to obtained the corresponding TFA salt of 3-(2-(2-aminoethoxy)ethoxy)-N-(2-(4-(2-(5-(3,5-dimethylisoxazol-4-yl)-1-(2-morpholinoethyl)-1H-benzo[d]imidazol-2- yl)ethyl)phenoxy)ethyl)propenamide (P300-PEG₂-NH₂. TFA), was obtained were used in next step without further purification. To a solution of corresponding TFA salt of 3-(2-(2-aminoethoxy)ethoxy)-N-(2-(4-(2-(5-(3,5-dimethylisoxazol-4-yl)-1-(2-morpholinoethyl)-1H-benzo[d]imidazol-2- yl)ethyl)phenoxy)ethyl)propenamide (5.0 mg, 0.0077 mmol, 1.0 eq) in DMF (1 mL), DIPEA (6.70 μL, 0.038 mmol, 5.0 eq.) and 2-(3-((R)-3-(3,4-dimethoxyphenyl)-1-(((S)-1-((S)-2-(3,4,5-trimethoxyphenyl)butanoyl)piperidine-2- carbonyl)oxy)propyl)phenoxy)acetic acid (synthesized as previously described2) (5.34 mg, 0.0077 mmol, 1 eq) was added followed by HATU (3.22 mg, 0.0085 mmol, 1.1 eq) were added at 0° C. and resulting mixture was stirred for 5 minutes the corresponding starting material was fully consumed (indicated by TLC). The crude mixture was diluted with cold water and extracted in ethyl acetate (20 mlL×3) then combined organic extract was dried over sodium sulfate, filtered, and concentrated under reduced pressure. The crude material was purified by PTLC (DCM/Methanol, 19:1) to obtain AceTAG-1 as a colorless sticky material (5.4 mg, 53%).

¹H NMR (600 MHz, CDCl₃): δ 7.55 (d, J=1.5 Hz, 1H), 7.29 (d, J=8.2 Hz, 1H), 7.25 (t, J=8.0 Hz, 0.25H), 7.14-7.05 (m, 4H), 7.03-6.98 (m, 1H), 6.94 (dt, J=7.6, 1.2 Hz, 0.23H), 6.83 (dd, J=2.6, 1.5 Hz, 0.22H), 6.80-6.77 (m, 0.27H), 6.77-6.74 (m, 2H), 6.73-6.71 (m, 0.36H), 6.70-6.68 (m, 2.6H), 6.67-6.63 (m, 1H), 6.62-6.57 (m, 3.3H), 6.35 (s, 0.4H), 6.34 (s, 2H), 5.72 (dd, J=7.8, 6.0 Hz, 0.2H), 5.55 (dd, J=8.3, 5.4 Hz, 1H), 5.41-5.33 (m, 1H), 4.59 (d, J=5.6 Hz, 0.2H), 4.44-4.38 (m, 2H), 4.09-4.04 (m, 2H), 3.96-3.91 (m, 2H), 3.84-3.74 (m, 8H), 3.74- 3.71 (m, 0.5H), 3.71 (s, 11H), 3.67-3.63 (m, 2.3H), 3.62-3.60 (m, 5H), 3.60-3.58 (m, 4.4H), 3.53-3.49 (m, 10H), 3.30-3.23 (m, 0.3H), 3.17 -3.14 (m, 2H), 3.12-3.06 (m, 2H), 2.73 (td, J=13.4, 3.0 Hz, 1H), 2.63-2.53 (m, 2.2H), 2.52-2.45 (m, 1.4H), 2.44-2.38 (m, 7H), 2.36 (s, 3.3H), 2.26-2.20 (s, 4.3H), 2.05-1.95 (m, 2.6H), 1.88-1.82 (m, 2H), 1.69-1.58 (m, 4H), 1.56-1.51 (m, 1.6H), 1.42-1.34 (m, 3H), 1.31 (t, J=7.4 Hz, 1H), 0.83 (t, J=7.3 Hz, 3H), 0.78 (t, J=7.3 Hz, 1.3H). Note: rotomeric isomers observed.

¹³C NMR (151 MHz, CDCl₃): δ 172.70, 172.38, 171.66, 171.59, 170.62, 168.34, 168.10, 165.06, 159.03, 157.41, 157.30, 157.24, 157.21, 155.38, 155.35, 153.50, 153.20, 148.97, 148.89, 147.52, 147.38, 142.99, 142.37, 141.89, 136.96, 136.62, 135.98, 135.34, 134.28, 133.43, 133.33, 133.28 130.16, 129.88, 129.44, 124.25, 123.46, 120.20, 120.12, 119.97, 119.91, 119.81, 117.11, 114.68, 114.14, 113.82, 113.70, 112.98, 111.71, 111.48, 111.28, 109.46, 104.97, 104.58, 75.66, 70.27, 70.13, 69.80, 67.39, 67.17, 66.84, 65.91, 60.91, 60.79, 57.64, 56.31, 55.97, 55.93, 55.88, 55.86, 55.76, 54.88, 54.06, 52.08, 51.24, 50.79, 43.49, 41.52, 39.70, 38.92, 38.85, 38.82, 38.27, 37.99, 36.89, 36.86, 32.94, 31.94, 31.50, 31.30, 29.84, 29.72, 29.34, 28.37, 26.81, 26.50, 25.33, 24.52, 22.71, 20.94, 20.76, 17.58, 17.06, 14.15, 12.74, 12.59, 11.61, 10.92, 10.24. Note: rotomeric isomers observed.

HRMS calculated for C₇₃H₉₄N₇O₁₆, 1324.6752 (M+H⁺), found 1324.6749.

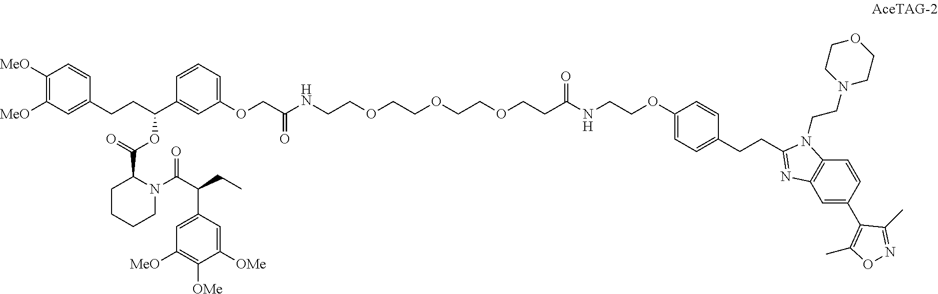 [SMILES: CC[C@H](C(=O)N1CCCC[C@H]1C(=O)O[C@H](CCc1ccc(OC)c(OC)c1)c1cccc(OCC(=O)NCCOCCOCCOCCC(=O)NCCOc2ccc(CCc3nc4cc(-c5c(C)noc5C)ccc4n3CCN3CCOCC3)cc2)c1)c1cc(OC)c(OC)c(OC)c1]

(R)-3-(3,4-dimethoxyphenyl)-1-(3-((18-(4-(2-(5-(3,5-dimethylisoxazol-4-yl)-1-(2-morpholinoethyl)-1H-benzo[d]imidazol-2-yl)ethyl)phenoxy)- 2,15-dioxo-6,9,12-trioxa-3,16-diazaoctadecyl)oxy)phenyl)propyl (S)-1-((S)-2-(3,4,5-trimethoxyphenyl)butanoyl)piperidine-2-carboxylate (AceTAG-2):

To a solution of tert-butyl (15-(4-(2-(5-(3,5-dimethylisoxazol-4-yl)-1-(2-morpholinoethyl)-1H-benzo[d]imidazol-2-yl)ethyl)phenoxy)-12-oxo-3,6,9- trioxa-13-azapentadecyl)carbamate (P300-PEG₃-NHBoc) (10 mg, 0.013 mmol) in DCM (1 mL), a solution of 20% TFA in DCM (1 mL) was added at 0° C. and resulting mixture was stirred at room temperature for 2 h. After completion (monitored by TLC), the reaction mixture was evaporated under reduced pressure to obtained the corresponding TFA salt of 3-(2-(2-(2-aminoethoxy)ethoxy)ethoxy)-N-(2-(4-(2-(5-(3,5-dimethylisoxazol-4-yl)-1-(2-morpholinoethyl)-1H-benzo[d]imidazol-2- yl)ethyl)phenoxy)ethyl)propenamide, was obtained were used in next step without further purification. To a solution of corresponding TFA salt of 3-(2-(2-(2-aminoethoxy)ethoxy)ethoxy)-N-(2-(4-(2-(5-(3,5-dimethylisoxazol-4-yl)-1-(2-morpholinoethyl)-1H-benzo[d]imidazol-2- yl)ethyl)phenoxy)ethyl)propanamide (6.0 mg, 0.0087 mmol, 1 eq.) in DMF (1 mL), DIPEA (7.52 μL, 0.043 mmol, 5 eq.) and 2-(3-((R)-3-(3,4-dimethoxyphenyl)-1-(((S)-1-((S)-2-(3,4,5-trimethoxyphenyl)butanoyl)piperidine-2-carbonyl)oxy)propyl)phenoxy)acetic acid (synthesized as previously described2) (6.0 mg, 0.0087 mmol, 1 eq) was added followed by HATU (3.16 mg, 0.0083 mmol, 1.1 eq) were added at 0° C. and resulting mixture was stirred for 5 minutes, the corresponding starting material was fully consumed (indicated by TLC). The crude mixture was diluted with cold water and extracted in ethyl acetate (20 mL×3) then combined organic extract was dried over sodium sulfate, filtered, and concentrated under reduced pressure. The crude material was purified by PTLC (DCM/Methanol, 19:1) to obtain AceTAG-2 as a colorless sticky material (5.6 mg, 48%).

¹H NMR (600 MHz, CDCl₃): δ 7.62 (d, J=1.4 Hz, 1H), 7.35 (d, J=8.2 Hz, 1H), 7.32 (t, J=7.9 Hz, 0.3H), 7.22-7.11 (m, 4H), 7.08 (d, J=5.9 Hz, 1H), 7.00 (dt, J=7.6, 1.2 Hz, 0.3H), 6.90 (d, J=2.4 Hz, 0.3H), 6.86 (dd, J=8.4, 2.7, Hz, 0.5H), 6.85-6.80 (m, 3H), 6.80-6.74 (m, 3H), 6.71-6.63 (m, 3H), 6.41 (s, 0.4H), 6.40 (s, 2H), 5.81-5.76 (m, 0.23H), 5.62 (dd, J=8.3, 5.4 Hz, 1H), 5.46-5.42 (m, 1H), 4.66-4.60 (m, 0.5H), 4.50-4.45 (m, 2.2H), 4.13 (t, J=6.8 Hz, 2H), 4.01 (t, J=5.4 Hz, 2H), 3.87-3.82 (m, 8.5H), 3.80-3.76 (m, 3.2H), 3.72-3.69 (m, 2.5H), 3.69-3.67 (m, 5H), 3.67-3.64 (m, 4.3H), 3.63-3.61 (m, 3H), 3.61-3.56 (m, 11.4H), 3.55-3.51 (m, 2.2H), 3.35-3.32 (m, 0.4H), 3.25-3.20 (m, 2H), 3.19-3.14 (m, 2.2H), 2.81-2.76 (m, 1H), 2.62 (t, J=6.9 Hz, 2H), 2.58-2.51 (m, 1.3H), 2.49-2.43 (m, 7H), 2.43-2.40 (m, 3.4H), 2.33-2.26 (m, 4H), 2.17 (s, 0.25H), 2.13-2.04 (m, 2H), 2.02-1.98 (m, 1H), 1.95-1.88 (m, 1H), 1.83-1.79 (m, 0.3H), 1.61-1.59 (m, 1.3H), 1.45-1.40 (m, 1H), 0.89 (t, J=7.3 Hz, 3H), 0.84 (t, J=7.3 Hz, 1H). Note: rotomeric isomers observed.

¹³C NMR (151 MHz, CDCl₃) δ 172.67, 172.36, 171.74, 171.69, 170.61, 170.49, 168.21, 168.00, 165.05, 159.03, 157.44, 157.31, 157.26, 155.38, 153.49, 153.20, 148.97, 148.89, 147.52, 147.37, 143.06, 142.34, 141.86, 136.95, 136.62, 135.99, 135.34, 134.30, 133.39, 133.34, 133.08, 130.14, 129.86, 129.42, 124.23, 123.44, 120.19, 120.12, 119.94, 119.77, 117.11, 114.68, 114.28, 113.94, 113.61, 112.88, 111.70, 111.60, 111.35, 111.28, 109.44, 104.97, 104.57, 75.66, 70.51, 70.37, 70.31, 70.27, 69.74, 67.42, 67.38, 67.20, 66.84, 66.80, 60.91, 60.79, 57.65, 56.31, 55.97, 55.93, 55.88, 55.86, 55.77, 54.07, 52.07, 51.24, 50.80, 43.48, 41.52, 39.69, 38.88, 38.83, 38.29, 38.00, 36.90, 32.94, 31.50, 31.29, 30.96, 29.87, 29.72, 28.38, 26.82, 25.34, 24.52, 22.71, 22.63, 20.95, 20.77, 12.74, 12.59, 11.61, 10.92. Note: rotomeric isomers observed.

HRMS calculated for C₇₅H₉₈N₇O₁₇, 1368.7014 (M+H⁺), found 1368.7018.

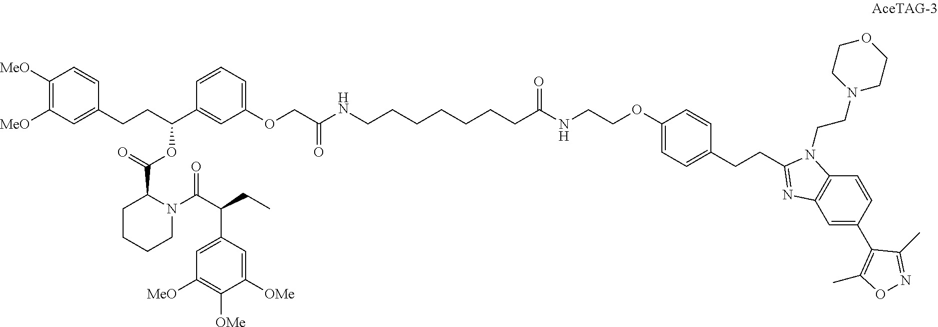 [SMILES: CC[C@H](C(=O)N1CCCC[C@H]1C(=O)O[C@H](CCc1ccc(OC)c(OC)c1)c1cccc(OCC(=O)NCCCCCCCC(=O)NCCOc2ccc(CCc3nc4cc(-c5c(C)noc5C)ccc4n3CCN3CCOCC3)cc2)c1)c1cc(OC)c(OC)c(OC)c1]

(R)-3-(3,4-dimethoxyphenyl)-1-(3-(2-((8-((2-(4-(2-(5-(3,5-dimethylisoxazol-4-yl)-1-(2-morpholinoethyl)-1H-benzo[d]imidazol-2- yl)ethyl)phenoxy)ethyl)amino)-8-oxooctyl)amino)-2-oxoethoxy)phenyl)propyl (S)-1-((S)-2-(3,4,5-trimethoxyphenyl)butanoyl)piperidine-2-carboxylate (AceTAG-3):

To a solution of tert-butyl (8-((2-(4-(2-(5-(3,5-dimethylisoxazol-4-yl)-1-(2-morpholinoethyl)-1H-benzo[d]imidazol-2-yl)ethyl)phenoxy)ethyl)amino)-8- oxooctyl)carbamate (P300-C7-NHBoc) (15mg, 0.020 mmol) in DCM (1 mL), a solution of 20% TFA in DCM (1 mL) was added at 0° C. and resulting mixture was stirred at room temperature for 2 h. After completion (monitored by TLC), the reaction mixture was evaporated under reduced pressure to obtain the corresponding TFA salt of 8-amino-N-(2-(4-(2-(5-(3,5-dimethylisoxazol-4-yl)-1-(2-morpholinoethyl)-1H-benzo[d]imidazol-2-yl)ethyl)phenoxy)ethyl)octanamide was obtained, were used in next step without further purification. To a solution of corresponding TFA salt of 8-amino-N-(2-(4-(2-(5-(3,5-dimethylisoxazol-4-yl)-1-(2-morpholinoethyl)-1H-benzo[d]imidazol-2-yl)ethyl)phenoxy)ethyl)octanamide (8.0 mg, 0.013 mmol, 1 eq.) in DCM (1 mL), DIPEA (7.52 μL, 0.043 mmol, 5 eq.) and 2-(3-((R)-3-(3,4-dimethoxyphenyl)-1-(((S)-1-((S)-2-(3,4,5-trimethoxyphenyl)butanoyl)piperidine-2-carbonyl)oxy)propyl)phenoxy)acetic acid (synthesized as previously described²) (8.8 mg, 0.013 mmol, 1 eq.) was added followed by EDC (3.65 mg, 0.019 mmol, 1.5 eq.) and HOBt (2.60 mg, 0.019 mmol, 1.5 eq.) were added at 0° C. and resulting mixture was stirred for 10 hours, the corresponding starting material was fully consumed (indicated by TLC). The crude mixture was diluted with cold water and extracted in DCM (20 mL×3) then combined organic extract was dried over sodium sulfate, filtered, and concentrated under reduced pressure. The crude material was purified by PTLC (DCM/Methanol, 19:1) to obtain AceTAG-3 as a colorless sticky material (7.8 mg, 47%).

¹H NMR (600 MHz, CDCl₃): δ 7.64 (s, 1H), 7.36 (s, 1H), 7.34-7.31 (m, 0.4H), 7.23-7.11 (m, 4H), 7.00 (d, J=7.6 Hz, 0.25H), 6.91 (s, 0.25H), 6.87-6.82 (m, 2.3H), 6.81-6.77 (m, 0.3H), 6.78-7.74 (m, 3H), 6.71-6.60 (m, 4H), 6.41 (s, 0.4H), 6.40 (s, 2H), 6.01-5.99 (m, 1H), 5.81-5.79 (m, 0.2H), 5.62 (dd, J=8.3, 5.4 Hz, 1H), 5.48-5.43 (m, 1H), 4.70-4.64 (m, 0.25H), 4.66-4.58 (m, 0.6H), 4.50-4.44 (m, 2H), 4.17-4.09 (m, 2H), 4.01 (t, J=5.1 Hz, 2H), 3.86-3.81 (m, 9H), 3.80-3.76 (s, 3.5H), 3.67-3.63 (m, 11H), 3.60-3.56 (m, 1H), 3.36-3.28 (m, 2.5H), 3.26-3.21 (m, 2.5H), 3.20-3.15 (m, 2.3H), 2.82-2.76 (m, 1H), 2.65-2.60 (m, J=6.9 Hz, 2.4H), 2.58-2.52 (m, 1.6H), 2.49-2.44 (m, 4.5H), 2.43-2.39 (m, 4.2H), 2.33-2.25 (m, 4.5H), 2.20-2.14 (m, 2.4H), 2.13-2.03 (m, 2.4H), 1.95-1.89 (m, 1H), 1.74-1.66 (m, 4H), 1.63-1.59 (m, 6H), 1.55-1.49 (m, 3H), 1.47-1.41 (m, 1.3H), 1.33-1.23 (m, 12H), 0.90 (t, J=7.3 Hz, 3H), 0.85 (t, J=7.3 Hz, 1.3H). Note: rotomeric isomers observed.

¹³C NMR (151 MHz, CDCl₃) δ 173.30, 172.70, 172.39, 170.66, 170.47, 168.03, 167.78, 165.06, 159.03, 157.43, 157.21, 155.39, 153.52, 153.22, 148.98, 148.91, 147.55, 147.40, 143.06, 142.39, 141.92, 137.00, 136.64, 136.01, 135.35, 134.31, 133.50, 133.37, 133.11, 130.17, 129.86, 129.47, 124.25, 123.46, 120.23, 120.15, 120.03, 119.95, 119.82, 117.12, 114.68, 113.83, 113.53, 113.09, 111.74, 111.65, 111.32, 109.45, 104.99, 104.61, 76.57, 67.39, 66.93, 66.85, 60.92, 60.80, 57.66, 56.33, 55.96, 55.88, 55.74, 54.08, 52.09, 51.26, 50.82, 43.49, 41.53, 39.69, 39.00, 38.92, 38.25, 37.97, 36.58, 32.96, 31.95, 31.31, 29.88, 29.73, 29.54, 29.09, 28.85, 28.40, 26.82, 26.63, 26.48, 25.53, 25.35, 24.54, 22.72, 20.93, 20.74, 14.16, 12.76, 12.61, 11.62, 10.93. Note: rotomeric isomers observed.

HRMS calculated for C₇₄H₉₆N₇O₁₄, 1306.7010 (M+H⁺), found 1306.7008.

### References

[1] G. A. Khoury, R. C. Baliban, C. A. Floudas, Scientific Reports 2011, 1, 90.
[2] T. Narita, B. T. Weinert, C. Choudhary, Nature Reviews Molecular Cell Biology 2019, 20, 156-174.
[3] G. Legube, D. Trouche, EMBO reports 2003, 4, 944-947.
[4] aA. Grabarska, J. J. Łuszczki, E. Nowosadzka, E. Gumbarewicz, W. Jeleniewicz, M. Dmoszyńska-Graniczka, K. Kowalczuk, K. Kupisz, K. Polberg, A. Stepulak, J Cancer 2017, 8, 19-28; bN. Al-Yacoub, L. F. Fecker, M. Möbs, M. Plötz, F. K. Braun, W. Sterry, J. Eberle, Journal of Investigative Dermatology 2012, 132, 2263-2274.
[5] B. T. Weinert, T. Narita, S. Satpathy, B. Srinivasan, B. K. Hansen, C. Schölz, W. B. Hamilton, B. E. Zucconi, W. W. Wang, W. R. Liu, J. M. Brickman, E. A. Kesicki, A. Lai, K. D. Bromberg, P. A. Cole, C. Choudhary, Cell 2018, 174, 231-244.e212.
[6] aD. A. Hay, O. Fedorov, S. Martin, D. C. Singleton, C. Tallant, C. Wells, S. Picaud, M. Philpott, O. P. Monteiro, C. M. Rogers, S. J. Conway, T. P. Rooney, A. Tumber, C. Yapp, P. Filippakopoulos, M. E. Bunnage, S. Muller, S. Knapp, C. J. Schofield, P. E. Brennan, J Am Chem Soc 2014, 136, 9308-9319; bT. Clackson, W. Yang, L. W. Rozamus, M. Hatada, J. F. Amara, C. T. Rollins, L. F. Stevenson, S. R. Magari, S. A. Wood, N. L. Courage, X. D. Lu, F. Cerasoli, M. Gilman, D. A. Holt, Proceedings of the National Academy of Sciences of the United States of America 1998, 95, 10437-10442.
[7] aB. M. Dancy, P. A. Cole, Chem Rev 2015, 115, 2419-2452; bT. Narita, B. T. Weinert, C. Choudhary, Nat Rev Mol Cell Biol 2019, 20, 156-174.
[8] aY. Tang, W. Zhao, Y. Chen, Y. Zhao, W. Gu, Cell 2008, 133, 612-626; bS. Timmermann, H. Lehrmann, A. Polesskaya, A. Harel-Bellan, Cellular and Molecular Life Sciences CMLS 2001, 58, 728-736.
[9] E. Szenker, D. Ray-Gallet, G. Almouzni, Cell Research 2011, 21, 421-434.
[10] B. Han, J Biol Chem 2020, 295, 15280-15291.
[11] aW. Gu, R. G. Roeder, Cell 1997, 90, 595-606; bS. M. Reed, D. E. Quelle, Cancers (Basel) 2014, 7, 30-69.
[12] aF. Mantovani, L. Collavin, G. Del Sal, Cell Death & Differentiation 2019, 26, 199-212; bA. B. Herrero, E. A. Rojas, I. Misiewicz-Krzeminska, P. Krzeminski, N. C. Gutiérrez, Int J Mol Sci 2016, 17, 2003.
[13] S. M. Sykes, H. S. Mellert, M. A. Holbert, K. Li, R. Marmorstein, W. S. Lane, S. B. McMahon, Mol Cell 2006, 24, 841-851.
[14] aN. A. Barlev, L. Liu, N. H. Chehab, K. Mansfield, K. G. Harris, T. D. Halazonetis, S. L. Berger, Molecular Cell 2001, 8, 1243-1254; bJ. Luo, M. Li, Y. Tang, M. Laszkowska, R. G. Roeder, W. Gu, Proceedings of the National Academy of Sciences of the United States of America 2004, 101, 2259-2264.
[15] Y. Zhao, S. Lu, L. Wu, G. Chai, H. Wang, Y. Chen, J. Sun, Y. Yu, W. Zhou, Q. Zheng, M. Wu, G. A. Otterson, W .-G. Zhu, Molecular and Cellular Biology 2006, 26, 2782-2790.
[16] S. Al Bitar, H. Gali-Muhtasib, Cancers (Basel) 2019, 11, 1475.
[17] aV. J. N. Bykov, S. E. Eriksson, J. Bianchi, K. G. Wiman, Nat Rev Cancer 2018, 18, 89-102; bA. Mogi, H. Kuwano, J Biomed Biotechnol 2011, 2011, 583929.
[18] aY. Liu, O. Tavana, W. Gu, J Mol Cell Biol 2019, 11, 564-577; bM. Li, J. Luo, C. L. Brooks, W. Gu, J Biol Chem 2002, 277, 50607-50611; cD. Wang, N. Kon, G. Lasso, L. Jiang, W. Leng, W .-G. Zhu, J. Qin, B. Honig, W. Gu, Nature 2016, 538, 118-122; dY. Tang, J. Luo, W. Zhang, W. Gu, Molecular Cell 2006, 24, 827-839; eS.-J. Wang, D. Li, Y. Ou, L. Jiang, Y. Chen, Y. Zhao, W. Gu, Cell Reports 2016, 17, 366-373; fT. Li, N. Kon, L. Jiang, M. Tan, T. Ludwig, Y. Zhao, R. Baer, W. Gu, Cell 2012, 149, 1269-1283.
[19] S. M. Reed, D. E. Quelle, Cancers (Basel) 2014, 7, 30-69.
[20] L. M. Zink, S. B. Hake, Curr Opin Genet Dev 2016, 37, 82-89.
[21] Z. Wang, C. Zang, J. A. Rosenfeld, D. E. Schones, A. Barski, S. Cuddapah, K. Cui, T. Y. Roh, W. Peng, M. Q. Zhang, K. Zhao, Nat Genet 2008, 40, 897-903.
[22] aB. T. Weinert, T. Narita, S. Satpathy, B. Srinivasan, B. K. Hansen, C. Scholz, W. B. Hamilton, B. E. Zucconi, W. W. Wang, W. R. Liu, J. M. Brickman, E. A. Kesicki, A. Lai, K. D. Bromberg, P. A. Cole, C. Choudhary, Cell 2018, 174, 231-244 e212; bR. Raisner, S. Kharbanda, L. Jin, E. Jeng, E. Chan, M. Merchant, P. M. Haverty, R. Bainer, T. Cheung, D. Arnott, E. M. Flynn, F. A. Romero, S. Magnuson, K. E. Gascoigne, Cell Rep 2018, 24, 1722-1729; cP. Sen, Y. Lan, C. Y. Li, S. Sidoli, G. Donahue, Z. Dou, B. Frederick, Q. Chen, L. J. Luense, B. A. Garcia, W. Dang, F. B. Johnson, P. D. Adams, D. C. Schultz, S. L. Berger, Mol Cell 2019, 73, 684-698 e688; dD. Bosnakovski, M. T. da Silva, S. T. Sunny, E. T. Ener, E. A. Toso, C. Yuan, Z. Cui, M. A. Walters, A. Jadhav, M. Kyba, Sci Adv 2019, 5, eaaw7781.
[23] D. A. Bose, G. Donahue, D. Reinberg, R. Shiekhattar, R. Bonasio, S. L. Berger, Cell 2017, 168, 135-149 e122.
[24] E. Michishita, R. A. McCord, E. Berber, M. Kioi, H. Padilla-Nash, M. Damian, P. Cheung, R. Kusumoto, T. L. A. Kawahara, J. C. Barrett, H. Y. Chang, V. A. Bohr, T. Ried, O. Gozani, K. F. Chua, Nature 2008, 452, 492-496.
[25] D. S. Cabianca, C. Muñoz-Jiménez, V. Kalck, D. Gaidatzis, J. Padeken, A. Seeber, P. Askjaer, S. M. Gasser, Nature 2019, 569, 734-739.
[26] aH. Yang, M. R. Green, Front Cell Dev Biol 2019, 7, 272; bM. G. Cardenas, E. Oswald, W. Yu, F. Xue, A. D. Mackerell, A. M. Melnick, Clinical Cancer Research 2017, 23, 885.
[27] aM. G. Cortiguera, L. Garcia-Gaipo, S. D. Wagner, J. Leon, A. Batlle-Lopez, M. D. Delgado, Sci Rep 2019, 9, 16495; bO. R. Bereshchenko, W. Gu, R. Dalla-Favera, Nat Genet 2002, 32, 606-613.
[28] aC. G. Parker, A. Galmozzi, Y. Wang, B. E. Correia, K. Sasaki, C. M. Joslyn, A. S. Kim, C. L. Cavallaro, R. M. Lawrence, S. R. Johnson, I. Narvaiza, E. Saez, B. F. Cravatt, Cell 2017, 168, 527-541 e529; bB. K. Erickson, C. M. Rose, C. R. Braun, A. R. Erickson, J. Knott, G. C. McAlister, M. Wuhr, J. A. Paulo, R. A. Everley, S. P. Gygi, Mol Cell 2017, 65, 361-370.
[29] A. Ito, T. Shimazu, S. Maeda, A. A. Shah, T. Tsunoda, S. I. Iemura, T. Natsume, T. Suzuki, H. Motohashi, M. Yamamoto, M. Yoshida, Science Signaling 2015, 8.
[30] Chen, L .; Fischle, W .; Verdin, E .; Greene, W. C. Duration of nuclear NF-kappaB action regulated by reversible acetylation. Science 2001, 293 (5535), 1653-1657.
[31] Reed, S. M.; Quelle, D. E. p53 Acetylation: Regulation and Consequences. Cancers 2015, 7(1), 30-69.

### Summary Statements

The inventions, examples, biological assays and results described and claimed herein have may attributes and embodiments include, but not limited to, those set forth or described or referenced in this application.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

Unless defined otherwise, all technical and scientific terms used herein have the same meaning as commonly understood by a person of ordinary skill in the art.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

It is also to be understood that as used herein and in the appended claims, the singular forms “a,” “an,” and “the” include plural reference unless the context clearly dictates otherwise, for example, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

The term and/or means both as well as one or the other as in A and/or B means A alone, B alone and A and B together.

1. A heterobifunctional composition comprising Formula I:
PBT-L-RBT (Formula I)
wherein PBT is a protein target binding moiety; L is an organic linker and RBT is a recruiter binding moiety for an acetylase enzyme.

2. A composition according to claim 1, wherein the PBT is a small organic molecule that is capable of binding a human protein of a biological pathway that is modified by lysine acetylation.

3. A composition according to claim 2, wherein the PBT is capable of binding the human protein with a Kd of no more than 10⁻⁴.

4. A composition according to claim 2, wherein the human protein is selected from the group consisting of an FKBP^{F36V} protein.

5. A composition according to claim 2, wherein the PBT capable of binding a human protein is

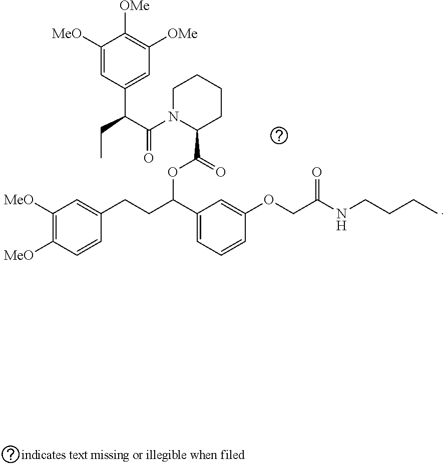 [SMILES: CCCCNC(=O)COc1cccc(C(CCc2ccc(OC)c(OC)c2)OC(=O)[C@@H]2CCCCN2C(=O)[C@@H](CC)c2cc(OC)c(OC)c(OC)c2)c1 ; "."]

6. A composition according to claim 1, wherein the RBT is an organic molecule capable of endogenously recruiting an acetylase enzyme and has a structure

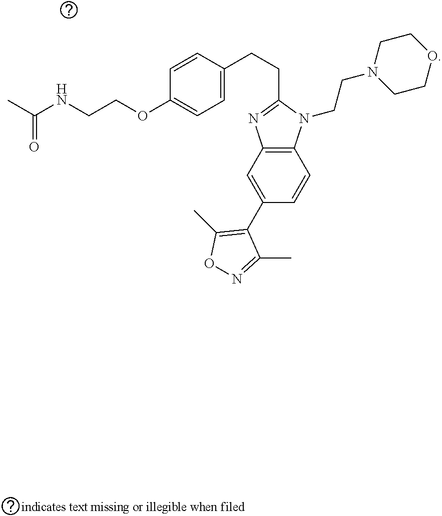 [SMILES: CC(=O)NCCOc1ccc(CCc2nc3cc(-c4c(C)noc4C)ccc3n2CCN2CCOCC2)cc1]

7. A composition according to claim 1, wherein the acetylase enzyme is p300CBP.

8. A composition according to claim 1, wherein L is a bivalent residue of an α-amino-polyethoxy-Ω-carboxylic acid or a bivalent residue of an α-amino-alkylenyl-Ω-carboxylic acid.

9. A composition according to claim 1, wherein PBT-L-RBT of Formula I is Formula II

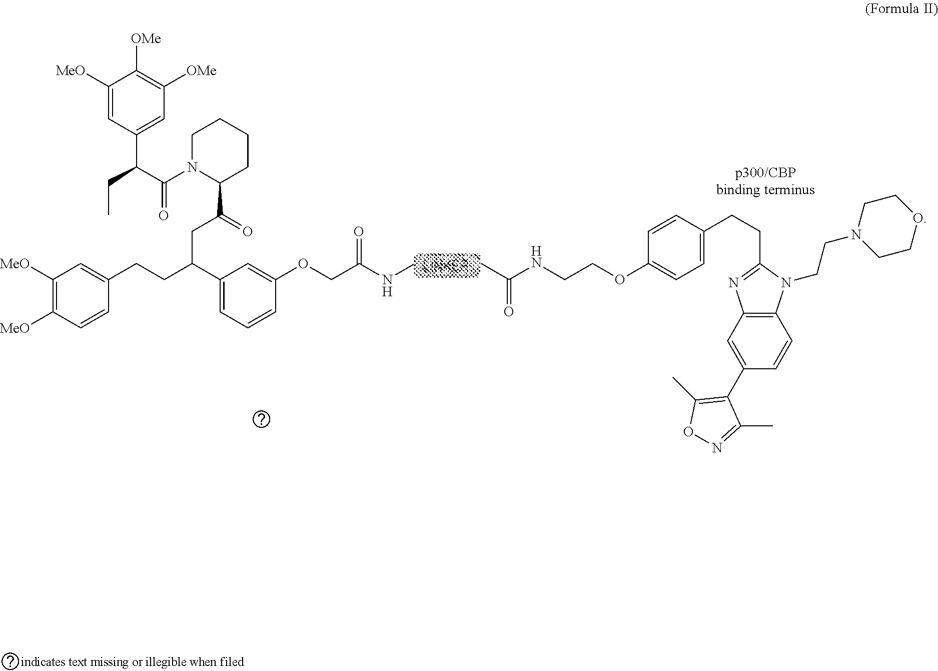 [SMILES: CC[C@H](C(=O)N1CCCC[C@H]1C(=O)CC(CCc1ccc(OC)c(OC)c1)c1cccc(OCC(=O)NC)c1)c1cc(OC)c(OC)c(OC)c1 ; CC(=O)NCCOc1ccc(CCc2nc3cc(-c4c(C)noc4C)ccc3n2CCN2CCOCC2)cc1]

10. A composition according to claim 9, wherein Formula II is selected from any one or more of

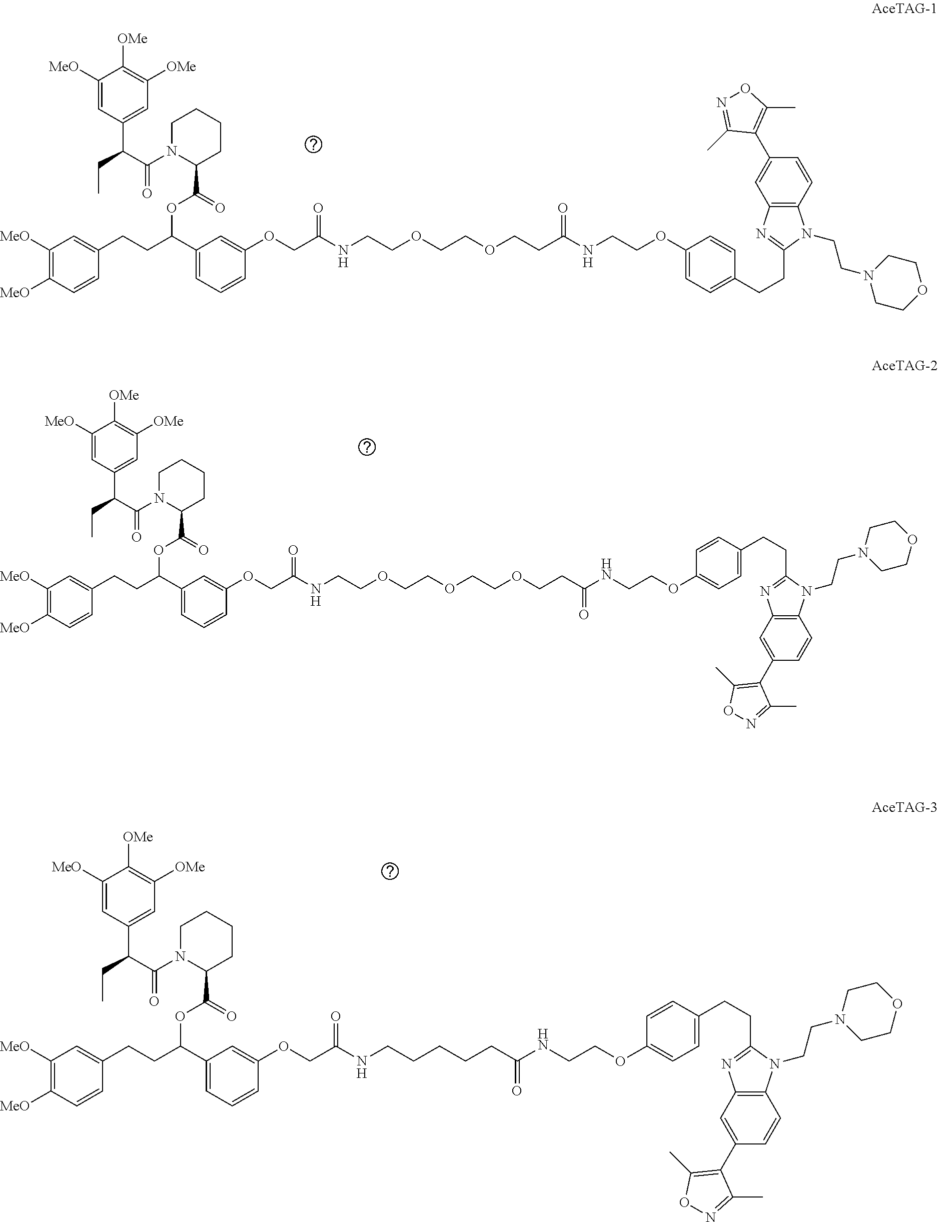 [SMILES: CC[C@H](C(=O)N1CCCC[C@H]1C(=O)OC(CCc1ccc(OC)c(OC)c1)c1cccc(OCC(=O)NCCOCCOCCC(=O)NCCOc2ccc(CCc3nc4cc(-c5c(C)noc5C)ccc4n3CCN3CCOCC3)cc2)c1)c1cc(OC)c(OC)c(OC)c1 ; CC[C@H](C(=O)N1CCCC[C@H]1C(=O)OC(CCc1ccc(OC)c(OC)c1)c1cccc(OCC(=O)NCCOCCOCCOCCC(=O)NCCOc2ccc(CCc3nc4cc(-c5c(C)noc5C)ccc4n3CCN3CCOCC3)cc2)c1)c1cc(OC)c(OC)c(OC)c1 ; CC[C@H](C(=O)N1CCCC[C@H]1C(=O)OC(CCc1ccc(OC)c(OC)c1)c1cccc(OCC(=O)NCCCCCC(=O)NCCOc2ccc(CCc3nc4cc(-c5c(C)noc5C)ccc4n3CCN3CCOCC3)cc2)c1)c1cc(OC)c(OC)c(OC)c1]

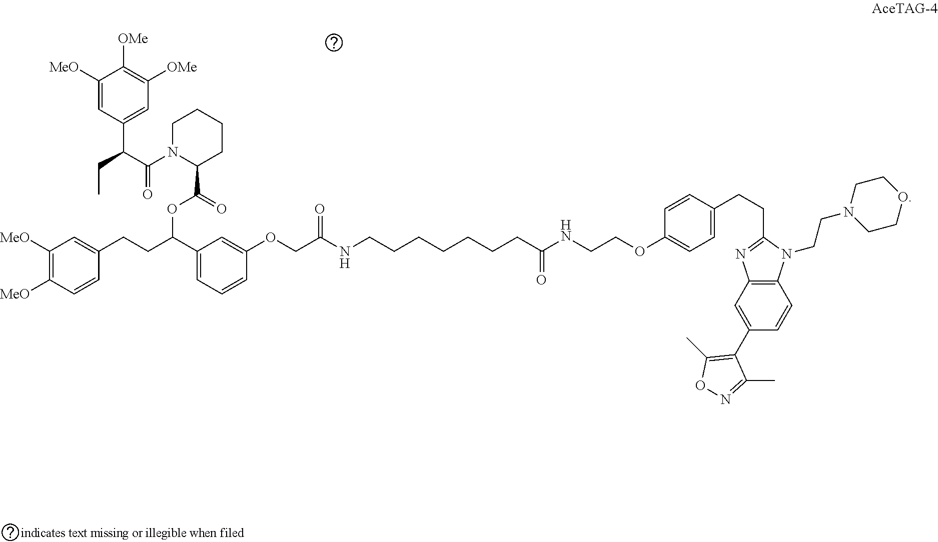 [SMILES: CC[C@H](C(=O)N1CCCC[C@H]1C(=O)OC(CCc1ccc(OC)c(OC)c1)c1cccc(OCC(=O)NCCCCCCCC(=O)NCCOc2ccc(CCc3nc4cc(-c5c(C)noc5C)ccc4n3CCN3CCOCC3)cc2)c1)c1cc(OC)c(OC)c(OC)c1]

11. A method for acetylation of an endogenous protein having lysine in its peptide sequence, comprising contacting the protein with the composition of claim 1 and an endogenous acetylation enzyme.

12. A method according to claim 11, wherein the endogenous protein is FKBP^{F36V} protein.

13. A method according to claim 11, wherein the endogenous acetylation enzyme is p300CBP.

14. A method according to claim 11, wherein the endogenous protein and endogenous acetylation enzyme are present in viable cells.

15. A method according to claim 14, wherein the viable cells are human cells.

16. A method according to claim 15, wherein the human cells are HeLa cells.

17. A method according to claim 16, wherein the HeLa cells are bioengineered to express FKP^{F36V} protein.

18. A method for modifying a protein of the p53 system of a viable cell, comprising contacting the viable cell with a composition of claim 1.

19. A method according to claim 18, wherein the modifying comprises acylation of at least one lysine residue of one or more proteins of the p53 system.

20. A method according to claim 18, wherein the viable cell is a human cancer cell.


SEQUENCE LISTING (10 sequences)

SEQ ID NO: 1 - DNA, 53 bp, Artificial sequence
  Synthetic sequence
ggggacaagtttgtacaaaaaagcaggcttcatggctcgtacaaagcagactg

SEQ ID NO: 2 - DNA, 50 bp, Artificial sequence
  Synthetic sequence
ggggaccactttgtacaagaaagctgggtcagcacgttctccacgtatgc

SEQ ID NO: 3 - DNA, 51 bp, Artificial sequence
  Synthetic sequence
ggggacaagtttgtacaaaaaagcaggcttcgaggagccgcagtcagatcc

SEQ ID NO: 4 - DNA, 51 bp, Artificial sequence
  Synthetic sequence
ggggaccactttgtacaagaaagctgggtctcagtctgagtcaggcccttc

SEQ ID NO: 5 - DNA, 50 bp, Artificial sequence
  Synthetic sequence
ggggacaagtttgtacaaaaaagcaggcttcatggaggagccgcagtcag

SEQ ID NO: 6 - DNA, 48 bp, Artificial sequence
  Synthetic sequence
ggggaccactttgtacaagaaagctgggtcgtctgagtcaggcccttc

SEQ ID NO: 7 - DNA, 55 bp, Artificial sequence
  Synthetic sequence
ggggacaagtttgtacaaaaaagcaggcttcgacgaactgttccccctcatcttc

SEQ ID NO: 8 - DNA, 55 bp, Artificial sequence
  Synthetic sequence
ggggaccactttgtacaagaaagctgggtcttaggagctgatctgactcagcagg

SEQ ID NO: 9 - DNA, 22 bp, Artificial sequence
  Synthetic sequence
tgttccgcattctgcaagcctc

SEQ ID NO: 10 - DNA, 25 bp, Artificial sequence
  Synthetic sequence
acaaaggcattaaagcagcgtatcc